(19) United States
(12) Patent Application Publication
(10) Pub. No.: US 2026/0310530 A1
(43) Pub. Date: Oct. 8, 2026

(54) CONNECTING APPLICATIONS WITH EXTERNAL SYSTEMS VIA A SINGLE COMMUNICATION CHANNEL

(71) Applicant: GE Precision Healthcare LLC, Waukesha, WI (US)

(72) Inventors: Angelo Fernandes, Bangalore (IN); Durga Phani Kanth Pilli, Bangalore (IN)

(21) Appl. No.: 19/170,142

(22) Filed: Apr. 4, 2025

(51) Int. Cl. G06F 9/54; H04L 9/40

(52) U.S. Cl. CPC G06F 9/547; H04L 63/02

(57) ABSTRACT

One or more systems, devices, computer program products and/or computer-implemented methods of use provided herein relate to connecting applications running in medical devices with external systems via a single communication channel. For example, according to an embodiment, a system is provided. The system can comprise a memory that can store computer-executable components. The system can further comprise a processor that can execute the computer-executable components stored in the memory, where the computer-executable components can comprise a core agent component that can access one or more requests from one or more agent applications. The computer-executable components can further comprise a core application component that can receive, via a communication channel, the one or more requests from the core agent component and that can transmit the one or more requests to one or more main applications external to the one or more agent applications.

### BACKGROUND

The subject disclosure relates to Internet of Things (IoT) systems and, more specifically, to connecting applications with external systems via a single communication channel.

### SUMMARY

The following presents a summary to provide a basic understanding of one or more embodiments described herein. This summary is not intended to identify key or critical elements, delineate scope of particular embodiments or scope of claims. Its sole purpose is to present concepts in a simplified form as a prelude to the more detailed description that is presented later. In one or more embodiments described herein, systems, computer-implemented methods, apparatus and/or computer program products that enable connecting applications with external systems via a single communication channel are discussed.

According to an embodiment, a system is provided. The system can comprise a memory that can store computer-executable components. The system can further comprise a processor that can execute the computer-executable components stored in the memory, where the computer-executable components can comprise a core agent component that can access one or more requests from one or more agent applications. The computer-executable components can further comprise a core application component that can receive, via a communication channel, the one or more requests from the core agent component and that can transmit the one or more requests to one or more main applications external to the one or more agent applications, wherein the communication channel can be based on an application protocol that can allow multiple requests to be transmitted from the one or more agent applications to the one or more main applications, via the core agent component and the core application component, by whitelisting only one Uniform Resource Locator (URL) on a firewall associated with the core application component.

According to another embodiment, a computer-implemented method is provided. The computer-implemented method can comprise accessing, by a system operatively coupled to a processor, via a core agent component, first data from one or more agent applications located within a medical device. The computer-implemented method can further comprise receiving, by the system, via a core application component, second data from the core agent component through a communication channel. The computer-implemented method can further comprise executing, by the system, via the core agent component and the core application component, one or more data transfer operations between the one or more agent applications and one or more main applications external to the one or more agent applications, in response to the receiving.

According to yet another embodiment, a computer program product is provided. The computer program product can comprise a non-transitory computer readable memory having program instructions embodied therewith, the program instructions executable by a processor to cause the processor to access, via a core agent component, one or more requests from one or more agent applications. The program instructions can be further executable by a processor to cause the processor to receive, via a core application component, the one or more requests from the core agent component through a communication channel. The program instructions can be further executable by a processor to cause the processor to transmit, via the core application component, the one or more requests to one or more main applications external to the one or more agent applications.

### BRIEF DESCRIPTION OF THE DRAWINGS

One or more embodiments are described below in the Detailed Description section with reference to the following drawings:

FIG. 1 illustrates a block diagram of an example, non-limiting system that can connect, via a single communication channel, one or more agent applications located within a medical device to one or more main applications external to the one or more agent applications, in accordance with one or more embodiments described herein.

FIG. 2 illustrates another block diagram of an example non-limiting system that can connect, via a single communication channel, one or more agent applications located within a medical device to one or more main applications external to the one or more agent applications, in accordance with one or more embodiments described herein.

FIG. 3 illustrates block diagrams of example, non-limiting systems that can connect, via multiple communication channels, one or more agent applications located within a medical device to one or more main applications external to the one or more agent applications.

FIG. 4 illustrates a block diagram of an example, non-limiting system that can connect, via a single communication channel, one or more agent applications located within a medical device to one or more main applications external to the one or more agent applications, in accordance with one or more embodiments described herein.

FIG. 5 illustrates a block diagram of an example non-limiting system that can transmit, via a single communication channel, a request from an agent application located within a medical device to a main application external to the agent application, in accordance with one or more embodiments described herein.

FIG. 6 illustrates a block diagram of an example, non-limiting system that can transfer, via a single communication channel, data between one or more agent applications and one or more main applications external to the one or more agent applications, in accordance with one or more embodiments described herein.

FIG. 7 illustrates a flow diagram of an example, non-limiting method that can upload, via a single communication channel, data from one or more agent applications to one or more main applications external to the one or more agent applications, in accordance with one or more embodiments described herein.

FIG. 8 illustrates a flow diagram of an example, non-limiting method that can download, via a single communication channel, data from one or more main applications to one or more agent applications external to the one or more main applications, in accordance with one or more embodiments described herein.

FIG. 9 illustrates a flow diagram of an example, non-limiting method that can transfer, via a server push capability of an application protocol, data from one or more main applications to one or more agent applications external to the one or more main applications, in accordance with one or more embodiments described herein.

FIG. 10 illustrates a block diagram of an example, non-limiting system that can transmit, via a stream prioritization capability of an application protocol, one or more requests from one or more agent applications located within a medical device to one or more main applications external to the one or more agent applications, in accordance with one or more embodiments described herein.

FIG. 11 illustrates flow diagrams of example, non-limiting methods that can connect agent applications located within a medical device to their corresponding main applications via a common communication channel, in accordance with one or more embodiments described herein.

FIG. 12 illustrates a block diagram of an example, non-limiting operating environment in which one or more embodiments described herein can be facilitated.

FIG. 13 illustrates an example networking environment operable to execute various implementations described herein.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

In the healthcare industry, medical devices such as imaging scanners can employ several software applications. It is desirable for software applications running on the medical devices to be lightweight in nature (e.g., dedicated to performing less intense computations) to reduce the number of computations and resources utilized by the medical device. Thus, such software applications are typically employed in a distributed architecture format, wherein a smaller portion of the software application (e.g., an agent application) can be located within the medical device, and the majority of the software application (e.g., a main application) can be located within an external environment. For example, the main application can reside in an external system. Cloud environments such as Amazon Web Services® (AWS®), Microsoft Azure®, Google Cloud®, etc. are popular choices of cloud environments in the industry, though other types of external systems can also be employed to run the main application. The agent application can act as a bridge between the medical device and the main application to realize a solution. Since a majority of the software application's main logic (e.g., the main application) can be external to the medical device, communication between the agent application running in the medical device and the main application is often achieved over Hypertext Transfer Protocol Secure (HTTPS) via a secured communication channel (also known as connection channel or common connectivity channel). Since the medical device is typically located in a medical facility within the medical facility's information technology (IT) network (e.g., a hospital's network, a clinic's network, etc.), the communication has to traverse the medical facility's firewall. Firewalls in medical facilities are typically highly restrictive to ensure privacy of sensitive medical data. For example, the endpoint of every communication is whitelisted in a medical facility's firewall for the communication between the agent application and the main application to be successfully established.

A commonly occurring scenario with medical devices is that the advent of cloud environments has enabled multiple agent applications to be run on a single medical device over a period of time. Running multiple agent applications on a medical device can involve whitelisting respective endpoints of main applications associated with respective software applications on a medical facility's firewall. Often, a main application can also comprise more than one endpoint that is to be whitelisted. Thus, the number of instances of whitelisting an endpoint on a firewall can be expected to increase as new software applications are installed in a medical device. The increased number of whitelisting instances can present challenges in terms of scaling multiple software applications being run on a medical device. For example, when a new software application is installed in the medical device either remotely or locally, the relevant project team at the concerned medical facility has to work with the medical facility's administration for the whitelisting to ensure that the new software application is successfully installed. This is a time-consuming, tedious and heavily collaborative process as it involves collaboration from multiple IT departments in the medical facility's organization. Additionally, onboarding (e.g., installing/adding) each new software application to the medical device can involve conducting a security review with the infrastructure team of the medical facility to demonstrate to the infrastructure team the flow of data out of the system. This can lead to a slower adoption rate of a software application, thereby impacting the revenue stream of the company supplying the software application. Consequently, the company's customers may assume that software applications are developed by the company in isolation, and the company does not have a unified view of a software application as an ecosystem. This can have a negative impact on the customer perception of the company. Nowadays, multiple artificial intelligence (AI) inferencing models are run on the cloud because AI inferencing can be computationally intense, and their counterpart agent applications are run on medical devices located in hospitals. Additionally, more medical facilities are adopting distributed architecture.

An existing technique known as tunnelling can encapsulate a network protocol within another network protocol to securely transmit data across a network and is commonly employed for secure communication, bypassing firewalls or enabling private network access over public infrastructures. However, existing tunnelling solutions for medical devices do not allow multiple applications (e.g., agent applications) running on a medical device to interact with their counterparts (e.g., main applications) residing within external systems by performing a single instance of whitelisting on a medical facility's firewall. Thus, more efficient solutions to implement distributed architectures for software applications employed by medical devices are desirable.

Various embodiments of the present disclosure can be implemented to produce a solution to these problems. Embodiments described herein include systems, computer-implemented methods, and computer program products that can connect applications running in medical devices to external systems via a single communication channel (also known as a common connectivity channel). The embodiments described herein can provide a unified way for agent applications running in a medical device to connect to their counterpart main applications by employing an application protocol (also known as communication protocol) that can leverage the Hypertext Transfer Protocol Version 2 (HTTP2). For example, in one or more embodiments, a core agent component (also known as core agent or core agent module) and a core application component (also known as core application or core application module) are provided, wherein the core agent component can be located within a medical device and the core application component can be located in an external system. The core agent component in combination with the core application component can provide a unified method for all agent applications employed by the medical device to communicate with the corresponding main applications located in the external system. The communication between the core agent component and the core application component can be securely established over HTTP2, which can natively support Transport Layer Security (TLS). TLS is a cryptographic protocol that can provide secure communication over a network.

In one or more embodiments, the core agent component can act as a gateway for outbound connections from the medical device. For example, the core agent component can access a request coming from one or more agent applications, wrap the request and forward the request to the core application component for further processing. The core agent component can also enable the upload and download of the files between the one or more agent applications and one or more main applications by collaborating with core application component. The core agent component in conjunction with the core application component can provide a layer of abstraction to allow the agent applications to be cloud agnostic. In one or more embodiments, the core application component can be a counterpart to the core agent component. For example, the core application component can unwrap the request received from the core agent component and delegate the request to the appropriate one or more main applications and to the appropriate resources within the one or more main applications. The core application component can comprise a resolver (also known as a resolver module or a router) that can resolve requests originating from the one or more agent applications and route the requests to the appropriate one or more main applications. The resolver can also assist with the upload and download of files between the one or more agent applications and the one or more main applications. For example, the resolver can temporarily hold/store a file in a queue while transporting the file between an agent application and a main application.

In one or more embodiments, the application protocol can comprise a unique design based on HTTP2, such that communication can be established between a plurality of agent applications installed in the medical device and their counterpart main applications located within an external environment by whitelisting only one endpoint. For example, as a result of the application protocol being built on HTTP2, communication between the core agent component and the core application component can be multiplexed, wherein a single Transmission Control Protocol (TCP) connection can be employed to send data from the agent applications to the main applications, thereby increasing the speed of data transfer. The agent applications can employ this method to transfer data of various formats and magnitudes to the main applications and to call the desirable Application Programming Interfaces (APIs) exposed in the main application to receive the desired response. In one or more embodiments, the server push and stream prioritization feature of HTTP2 can also be beneficially employed, as described with reference to one or more figures.

It should be further appreciated that throughout this specification, the term ‘agent application’ has been used to describe a portion of a software application employed within a medical device, and the term ‘main application’ has been used to describe a portion of a software application employed within an environment external to the medical device.

Embodiments of the present disclosure can provide the following business benefits:
Simplified services: Agent applications can be added to medical devices faster via a single registration for multiple agent applications and whitelisting a single endpoint.
Scalability: New capabilities (e.g., agent applications) can be added to a medical device without involving registration and whitelisting.
Simplified security checklists: Common customer security checklists can be provided for agent applications due to a single communication channel being opened to the outside world for all agent applications. On the contrary, in existing solutions, onboarding each agent application onto a medical device can involve addressing a separate security checklist and security questionnaire.
Cloud portability: Main applications can be migrated (e.g., moved) to a different cloud without applying any changes to or updating (e.g., rewiring or reprogramming) the agent applications and the firewall whitelisting criteria.

Embodiments of the present disclosure can provide the following customer benefits:
Simplified IT: The involvement of IT administrators per agent application employed in a medical device can be reduced due to only one endpoint being whitelisted on a medical facility's firewall for multiple agent applications.
Zero downtime: Since new agent applications can be added to a medical device without registration, the downtime involved in adding the new agent applications can also be eliminated. Contrary to existing solutions, the downtime experienced by end entities (e.g., hardware, software, machines, AI, neural networks and/or users) can be reduced.
Easy network monitoring (NW): An end entity (e.g., hardware, software, machine, AI, neural network and/or user) can monitor traffic from multiple agent applications by monitoring a single endpoint.
Unified security views: An end entity (e.g., hardware, software, machine, AI, neural network and/or user) can have a simplified security and connectivity view of multiple agent applications running on a medical device having outbound connectivity. Thus, the end entity has to review a single pipeline as opposed to multiple pipelines of data.

It should be appreciated that the embodiments of the preset disclosure are not limited to medical devices or to the healthcare industry. The framework for aggregating connections and transmitting data of various types between agent applications located in devices and main applications external to the agent applications can be employed in multiple devices and in a variety of industries. Embodiments of the present disclosure can be also employed to build a platform, and a variety of different applications can be developed based on the platform.

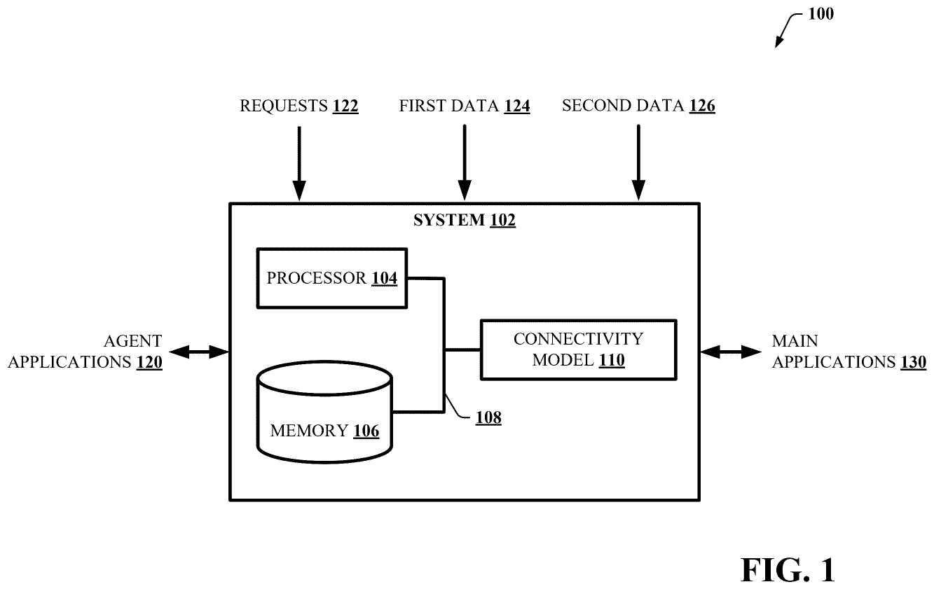

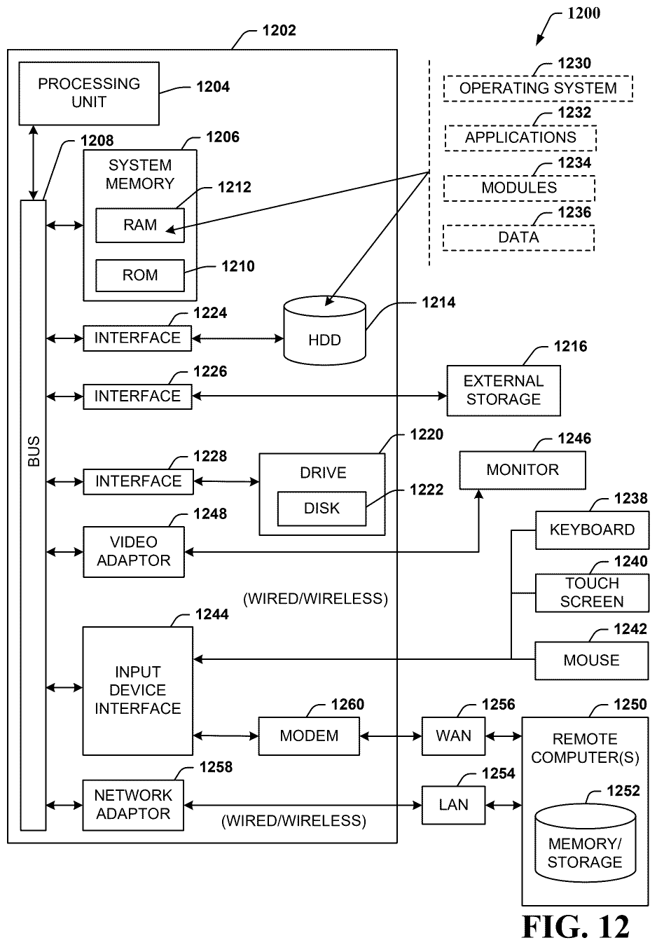

The embodiments depicted in one or more figures described herein are for illustration only, and as such, the architecture of embodiments is not limited to the systems, devices and/or components depicted therein, nor to any particular order, connection and/or coupling of systems, devices and/or components depicted therein. For example, in one or more embodiments, the non-limiting systems described herein, such as non-limiting system 100 as illustrated at FIG. 1, and/or systems thereof, can further comprise, be associated with and/or be coupled to one or more computer and/or computing-based elements described herein with reference to an operating environment, such as the operating environment 1200 illustrated at FIG. 12. For example, non-limiting system 100 can be associated with, such as accessible via, a computing environment 1200 described below with reference to FIG. 12, such that aspects of processing can be distributed between non-limiting system 100 and the computing environment 1200. In one or more described embodiments, computer and/or computing-based elements can be used in connection with implementing one or more of the systems, devices, components and/or computer-implemented operations shown and/or described in connection with FIG. 1 and/or with other figures described herein.

For simplicity of explanation, the computer-implemented and non-computer-implemented methodologies provided herein are depicted and/or described as a series of acts. It is to be understood that the subject innovation is not limited by the acts illustrated and/or by the order of acts, for example acts can occur in one or more orders and/or concurrently, and with other acts not presented and described herein. Furthermore, not all illustrated acts can be utilized to implement the computer-implemented and non-computer-implemented methodologies in accordance with the described subject matter. Additionally, the computer-implemented methodologies described hereinafter and throughout this specification are capable of being stored on an article of manufacture to enable transporting and transferring the computer-implemented methodologies to computers. The term article of manufacture, as used herein, is intended to encompass a computer program accessible from any computer-readable device or storage media.

The systems and/or devices have been (and/or will be further) described herein with respect to interaction between one or more components. Such systems and/or components can include those components or sub-components specified therein, one or more of the specified components and/or sub-components, and/or additional components. Sub-components can be implemented as components communicatively coupled to other components rather than included within parent components. One or more components and/or sub-components can be combined into a single component providing aggregate functionality. The components can interact with one or more other components not specifically described herein for the sake of brevity, but known by those of skill in the art.

FIG. 1 illustrates a block diagram of an example, non-limiting system 100 that can connect, via a single communication channel, one or more agent applications located within a medical device to one or more main applications external to the one or more agent applications, in accordance with one or more embodiments described herein.

Non-limiting system 100 and/or the components of non-limiting system 100 can be employed to use hardware and/or software to solve problems that are highly technical in nature (e.g., related to distributed architectures, network protocols, data transfer, etc.), that are not abstract and that cannot be performed as a set of mental acts by a human. Further, some of the processes performed may be performed by specialized computers for conducting defined tasks related to connecting applications with external systems via a single communication channel. Non-limiting system 100 and/or components of non-limiting system can be employed to solve new problems that arise through advancements in technologies mentioned above and/or the like. Non-limiting system 100 can provide technical improvements to software architectures by improving the accuracy of software applications in a distributed architecture, increasing the speed of data transmission between the software applications, increasing the speed of installing new applications in devices and reducing the system downtime involved in the installation.

In one or more embodiments, non-limiting system 100 can be employed to onboard new software products within a medical device remotely and without the involvement of field engineers (FEs) in the field during the onboarding. This can reduce travel and operational expenses and generate higher profits for companies developing the software products and make the process of familiarizing customers with the new software application faster. For example, as discussed elsewhere herein, non-limiting system 100 can provide a plug-and-play capability to distributed architectures, eliminating the issue of whitelisting multiple endpoints and increasing the speed of revenue collection and order fulfillment for a software development company when onboarding new software applications. Additionally, the design of the application protocol referenced in various embodiments herein can provide a faster, more accurate, safer (e.g., due to being based on HTTP2) and inexpensive system for communication between agent applications and main applications in a distributed architecture. Non-limiting system 100 can also allow a software development company to onboard more agent applications to a medical device faster.

In one or more embodiments, non-limiting system 100 can comprise system 102. Discussion turns briefly to processor 104, memory 106 and bus 108 of system 102. For example, in one or more embodiments, system 102 can comprise processor 104 (e.g., computer processing unit, microprocessor, classical processor, and/or like processor). In one or more embodiments, a component associated with system 102, as described herein with or without reference to the one or more figures of the one or more embodiments, can comprise one or more computer and/or machine readable, writable and/or executable components and/or instructions that can be executed by processor 104 to enable performance of one or more processes defined by such component(s) and/or instruction(s).

In one or more embodiments, system 102 can comprise a computer-readable memory (e.g., memory 106) that can be operably connected to processor 104. Memory 106 can store computer-executable instructions that, upon execution by processor 104, can cause processor 104 and/or one or more other components of system 102 (e.g., connectivity model 110, core agent component 202, core application component 204, application addition component 206 and/or migration component 208) to perform one or more actions. In one or more embodiments, memory 106 can store computer-executable components (e.g., connectivity model 110, core agent component 202, core application component 204, application addition component 206 and/or migration component 208). In one or more embodiments, one or more of the components of system 102 can reside in the cloud, and/or can reside locally in a local computing environment (e.g., at a specified location(s)).

Non-limiting system 100 and/or a component thereof as described herein, can be communicatively, electrically, operatively, optically and/or otherwise coupled to one another via bus 108. Bus 108 can comprise one or more of a memory bus, memory controller, peripheral bus, external bus, local bus, and/or another type of bus that can employ one or more bus architectures. One or more of these examples of bus 108 can be employed. In one or more embodiments, non-limiting system 100 can be coupled (e.g., communicatively, electrically, operatively, optically and/or via like function) to one or more external systems (e.g., a non-illustrated electrical output production system, one or more output targets, an output target controller and/or the like), sources and/or devices (e.g., classical computing devices, communication devices and/or like devices), such as via a network.

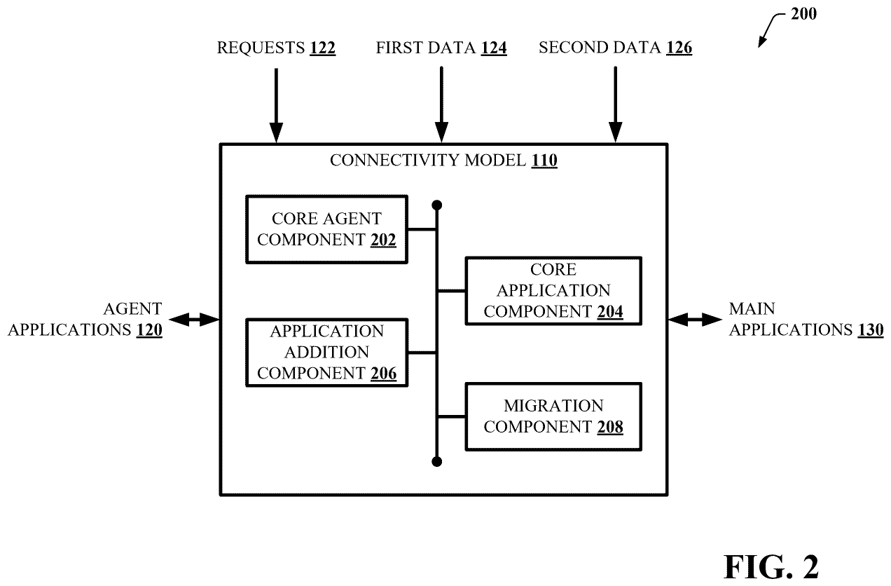

In one or more embodiments, system 102 can comprise connectivity model 110. As illustrated in FIG. 2, connectivity model 110 can comprise core agent component 202, core application component 204, application addition component 206 and migration component 208. In one or more embodiments, agent applications 120 can comprise one or more agent applications located within a medical device (e.g., a medical imaging device or another type of medical device), and main applications 130 can comprise one or more main applications external to agent applications 120. For example, agent applications 120 can be employed within a Magnetic Resonance Imaging (MRI) scanner, a Computer Tomography (CT) scanner or another medical device, and main applications 130 can be located within a cloud environment or another environment external to the medical device comprising agent applications 120. Additionally, one or more main applications of main applications 130 can be counterparts to one or more agent applications of agent applications 120. For example, an agent application of agent applications 120 and a corresponding main application of main applications 130 can represent a distributed architecture format of a software application employed by the medical device, wherein the agent application located within the medical device can perform lightweight computations, whereas the main application located within the cloud environment can perform the bulk of the computations associated with the software application. As such, one or more agent applications of agent applications 120 can correspond to one or more main applications of main applications of 130.

In one or more embodiments, connectivity model 110 can employ a single communication channel to transmit data and requests between agent applications 120 and main applications 130. For example, in one or more embodiments, one or more agent applications of agent applications 120 can generate requests 122 and transmit (e.g., send/forward) requests 122 to core agent component 202. Requests 122 can comprise one or more requests. Core agent component 202 can access requests 122 from agent applications 120, and core agent component 202 can transmit requests 122 to core application component 204 via the communication channel. Core application component 204 can receive requests 122 from core agent component 202 via the communication channel, and core application component 204 can transmit requests 122 to main applications 130. For example, upon receiving requests 122 from the one or more agent applications, core agent component 202 can wrap requests 122, wherein wrapping requests 122 can generate a wrapped request. Core agent component 202 can forward the wrapped request to core application component 204 via the communication channel. Core application component 204 can access the wrapped request and unwrap the wrapped request into requests 122 and route requests 122 to one or more main applications of main applications 130 via a resolver. For example, core application component 204 can forward respective requests comprised in requests 122 to the appropriate one or more main applications comprised in main applications 130. For example, core application component 204 can route a request originating from an agent application to the corresponding main application. In one or more embodiments, upon forwarding requests 122 to main applications 130, core application component 204 can access feedback from main applications 130, wherein the feedback is associated with routing requests 122, and core application component 204 can transmit the feedback to the one or more agent applications via core agent component 202.

In one or more embodiments, agent applications 120 can generate a variety of requests, and each request can also comprise data associated with the request. For example, agent application A can be one of the agent applications comprised in agent applications 120, and main application A can be one of the main applications comprised in main applications 130. Agent application A can have the identity app_id_A, and agent application A can intend to communicate with its counterpart, main application A. Accordingly, agent application A can interact with core agent component 202 with the intent to initiate communication with main application A. For example, agent application A can transmit a request to core agent component 202, wherein the request can comprise the main application ID, resource type, resource name, resource variables, resource method, payload data and payload data type. The resource type can typically comprise the resource classes exposed in main application A, for example, Object_Store, API, Notification, Queue, Security_Token, User_Mgmt, AI_Inferencing, etc. The resource method can typically comprise Hypertext Transfer Protocol (HTTP) methods like GET, POST, PUT, PATCH, DELETE, etc. The payload data type can comprise the data type of the payload that can be embedded in the request by agent application A. For example, the payload data type can have a JavaScript Object Notation (JSON) format, a Flat-file format, an Extensible Markup Language (XML) format, a Binary Large Object (BLOB) format, a JPEG image (JPG) format, Portable Network Graphics (PNG) format or another suitable format. Agent application A can also transmit an optional callback URL to core agent component 202, wherein the callback URL can be employed by core agent component 202 to call back agent application A, for example, in case of an asynchronous communication.

In one or more embodiments, core agent component 202 can act as a gateway internal to the medical device, and core application component 204 can act as a gateway internal to the external environment. For example, core agent component 202 can be located in the medical device comprising agent applications 120, and core application component 204 can be located in the external environment comprising main applications 130.

In one or more embodiments, agent applications 120 can interact with core agent component 202, and main applications 130 can interact with core application component 204 via service discovery. In software architecture, applications can interact via service discovery by dynamically locating and communicating with services in a distributed system. This is crucial in microservices architectures where services can frequently change locations due to scaling or failures. An interaction between applications via service discovery can typically comprise the following steps:
1. Service registration: When a service begins, it registers its metadata (such as name, Internet Protocol (IP) address, and port) with a service registry (e.g., Kubernetes Service Discovery, etc.).
2. Service discovery: Other applications query the service registry to find available instances of the desired service. The discovery can be client-side discovery, wherein the client application queries the registry and selects an instance to communicate with, or a server-side discovery, wherein a load balancer or proxy (e.g., Envoy or Kubernetes' kube-proxy) directs requests to an available instance.
3. Service communication: Once discovered, services communicate via protocols like HTTP, messaging queues, etc.
4. Health monitoring and deregistration: The registry continuously checks service health and removes unhealthy instances.

This process can ensure scalability, fault tolerance, and efficient service interaction in dynamic environments. In one or more embodiments, agent applications comprised within agent applications 120 as well as new agent applications added to agent applications 120 can similarly employ service discovery to automatically recognize and interact with core agent component 202. For example, the service name associated with core agent component 202 can be well known to the medical device comprising agent applications 120, and a new agent application onboarded to the medical device can automatically recognize and interact with the service name associated with core agent component 202. Main applications 130 can similarly employ service discovery to interact with core application component 204.

In one or more embodiments, the medical device comprising agent applications 120 and core agent component 202 can be configured to prevent agent applications 120 from bypassing core agent component 202, such that the medical device can transmit and receive requests and data only through core agent component 202. For example, the medical device can comprise an internal firewall that can be configured by an entity (e.g., hardware, software, machine, AI, neural network and/or user) to accept outbound traffic only from core agent component 202. For example, core agent component 202 can have a well-known name or process identifier (ID) that can be configured by the entity (e.g., hardware, software, machine, AI, neural network and/or user) as a rule inside the firewall of the medical device.

In one or more embodiments, the communication channel employed to transfer data and requests between agent applications 120 and main applications 130 can be based on an application protocol. The application protocol can allow agent applications 120 and main applications 130 to efficiently interact with each other via the communication channel and transmitting requests 122 and data over the communication channel can increase speed of transmission of the requests and simplify troubleshooting of connectivity issues between agent applications 120 and main applications 130. For example, the application protocol can allow agent applications 120 to interact with main applications 130 via the communication channel by wrapping communications (e.g., requests 122) originating from agent applications 120 to main applications 130. Additionally, the application protocol can allow a plurality of requests comprised in requests 122 to be transmitted by core agent component 202 from one or more agent applications of agent applications 120 to one or more main applications of main applications 130, via core application component 204, by whitelisting a Uniform Resource Locator (URL) (as opposed to multiple URLs) on a firewall associated with core application component 204. Whitelisting a URL on a firewall can allow specific web addresses (URLs) to bypass firewall restrictions, ensuring that only approved websites can be accessed while other websites are blocked. As a result, new agent applications can be added to agent applications 120, and one or more main applications of main applications 130 can be migrated to different external environments efficiently. For example, application addition component 206 can add at least one new agent application to the medical device, wherein adding the at least one new agent application to the medical device does not involve updating the firewall and application downtime. Likewise, main applications 130 can be located in a first external system, and migration component 208 can migrate at least one main application of main applications 130 from the first external system to a second external system, wherein migrating the at least one main application to the second external system does not involve updating the firewall and application downtime. In one or more embodiments, the URL can also be employable by an end entity (e.g., hardware, software, machine, AI, neural network and/or user) to monitor traffic from the medical device and one or more additional medical devices by monitoring only the traffic associated with the URL.

In an embodiment, the application protocol can be based on HTTP2. HTTP2 can deliver better performance compared to HTTP, ensuring a defined quality of service according to industry standards. Additionally, HTTP2 can comprise server push and stream prioritization features that can provide a server push capability (or functionality) and a stream prioritization capability (or functionality) to the application protocol, as described at least by paragraphs [0062] to [0069]:

### Server Push Capability

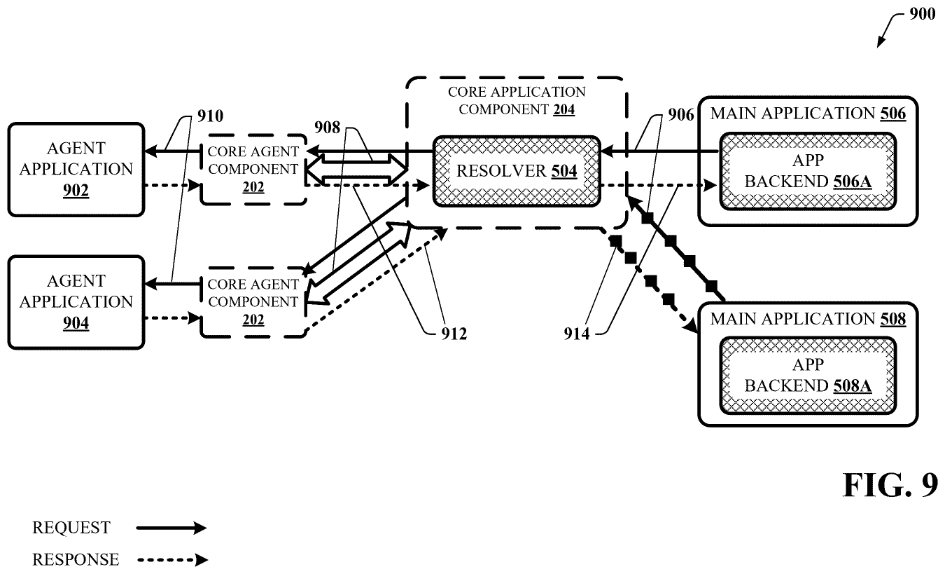

Server push is a feature in HTTP2, wherein a server (e.g., a main application) can proactively push resources onto a client (e.g., an agent application) before the client requests the resources. In one or more embodiments, core application component 204 can transmit data from one or more main applications of main applications 130 to one or more agent applications of agent applications 120, via core agent component 202, by employing the server push capability of the application protocol. It should be noted, however, that the originator of the communication channel should be an agent application comprised within agent applications 120 even when employing the server push capability of the application protocol to address security concerns and prevent unauthorized inbound connections to the medical device. Once a connection is established/opened between core agent component 202 and core application component 204, the server push capability can enable the one or more main applications (e.g., servers) to communicate with core agent component 202. For example, agent applications A1, A2 and A3 can represent some one of the agent applications comprised in agent applications 120, and main application A can be one of the main applications comprised in main applications 130. Main application A can have the intention to communicate with agent applications A1, A2 and A3. Accordingly, main application A can interact with core application component 204 with the intent to communicate with agent applications A1, A2 and A3. For example, main application A can transmit a request to core application component 204, wherein the request can comprise agent application identifiers (IDs), payload data and payload data type. Main application A can also include an optional callback URL in the request, and core application component 204 can employ the callback URL to call back main application A, for example, in case of an asynchronous communication. Upon receiving the request from main application A, core application component 204 can forward the payload associated with the payload data to core agent component 202, and core agent component 202 can transmit the payload to agent applications A1, A2 and A3. Additionally, core agent component 202 can transmit feedback to core application component 204, wherein the feedback can be indicative of a successful delivery of the payload to agent applications A1, A2 and A3, and core application component 204 can transmit the feedback to main application A. Additional embodiments related to the server push capability of the application protocol are described with reference to FIG. 9.

Listed below are some examples of requests that can be generated by a main application of main applications 130 and transmitted to one or more agent applications of agent applications 120 via the server push capability:

If a main application wants to transmit JSON data to one or more agent applications, the main application can transmit the following information to core application component 204:
{
“agentApplicationIDs”: [“app_id_A1”, “app_id_A2”, . . . ],
“payloadData”: “{“PAYLOAD_JSON”}”,
“payloadDataType”: “JSON”
}

If a main application wants to transmit Flat data to one or more agent applications, the main application can transmit the following information to core application component 204:
{
“agentApplicationIDs”: [“app_id_A1”, “app_id_A2”, . . . ],
“payloadData”: “12,45,22”,
“payloadDataType”: “flat”
}

If a main application wants to transmit data to one or more agent applications and receive a notification indicative of a successful delivery, the main application can transmit the following information to core application component 204:
{
“agentApplicationIDs”: [“app_id_A1”, “app_id_A2”, . . . ],
“payloadData”: “12,45,22”,
“payloadDataType”: “flat”,
“callBackURL”: “https://main_app_url/callback_url”
}

### Stream Prioritization

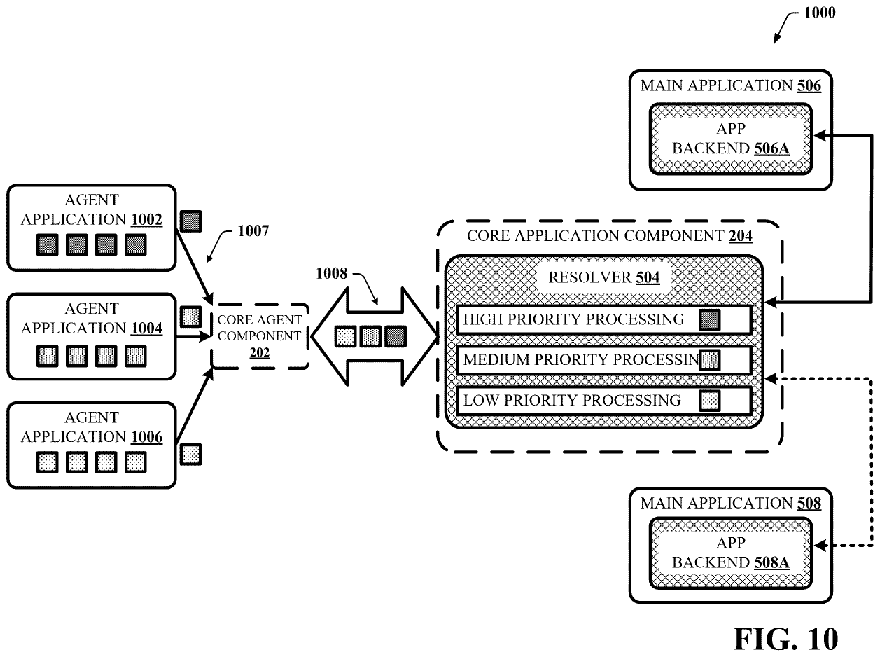

Stream prioritization is a feature in HTTP2 that allows clients (e.g., agent applications) to assign priority levels to different streams (e.g., requests generated by the agent applications) so that high priority resources can be loaded first. In one or more embodiments, core agent component 202 can prioritize transmission of respective requests comprised in requests 122 by employing a stream prioritization capability of the application protocol. For example, by leveraging the stream prioritization capability provided by HTTP2, a first agent application comprised within agent applications 120 can assign, based on the priority or level of criticality associated with the first agent application, a first priority (e.g., high, medium, low) to a stream (e.g., a request) transmitted by the first agent application to core agent component 202. Similarly, a second agent application comprised within agent applications 120 can assign, based on the priority or level of criticality associated with the second agent application, a second priority (e.g., high, medium, low) to a stream (e.g., a request) transmitted by the second agent application to core agent component 202, wherein the second priority can be different than the first priority. As a result, core application component 204 process the first and second streams according to their respective priorities by employing the stream prioritization capability of the application protocol. For example, if the first stream has a low priority and the second stream has a high priority, core application component 204 can process the second stream prior to processing the first stream. Additional embodiments related to the stream prioritization capability of the application protocol are described with reference to FIG. 10.

In one or more embodiments, the application protocol can also be employed by connectivity model 110 to update one or more agent applications comprised in agent applications 120 or one or more main applications comprised in main applications 130. However, such updates can only be performed after performing the applicable checks and balances and while remaining compliant with the regulations applicable to the industry.

Recall that agent applications 120 can comprise one or more agent applications located within a medical device, and main applications 130 can comprise one or more main applications located externally to agent applications 120. In one or more embodiments, connectivity model 110 can also allow files to be uploaded and downloaded, via the communication channel based on the application protocol based on HTTP2, between agent applications 120 and main applications 130. For example, in one or more embodiments, core agent component 202 can access first data 124 from agent applications 120, and in response to receiving first data 124, core agent component 202 can transmit second data 126 to core application component 204 via the communication channel. Core application component 204 can receive second data 126 from core agent component 202, and in response to receiving second data 126, core agent component 202 and core application component 204 can execute one or more data transfer operations between agent applications 120 and main applications 130.

For example, in an embodiment, first data 124 can comprise an intent of one or more agent applications comprised in agent applications 120 to upload a payload to one or more main applications comprised in main applications 130, the payload and a callback URL. Additionally, second data 126 can comprise the payload, and the one or more data transfer operations can comprise an upload operation to transfer the payload from the one or more agent applications to the one or more main applications. For example, upon receiving first data 124 comprising the intent of one or more agent applications to upload a payload to the one or more main applications, the payload and a callback URL, core agent component 202 can initiate an upload operation by generating second data 126 comprising the payload and transmitting second data 126 to core application component 204. That is, core agent component 202 can upload the payload to core application component 204. Specifically, core agent component 202 can upload the payload to a first bucket within core application component 204. In response to receiving the payload, core application component 204 can upload the payload, via a resolver, to the one or more main applications. Specifically, core application component 204 can upload the payload to a second bucket within main applications 130. Thereafter, core application component 204 can generate, via the resolver, feedback indicating the status of the upload operation. The feedback can be deposited by the resolver as a message in a queue within core application component 204. Core agent component 202 can access the feedback and transmit the feedback from the queue to the one or more agent applications via the callback URL.

In another embodiment, first data 124 can comprise an intent of one or more agent applications of agent applications 120 to download a payload from one or more main applications of main applications 130 and a callback URL. Additionally, second data 126 can comprise a download request, and the one or more data transfer operations can comprise a download operation to transfer the payload from the one or more main applications to the one or more agent applications. For example, upon receiving first data 124 comprising the intent of one or more agent applications to download a payload from the one or more main applications and a callback URL, core agent component 202 can initiate a download operation by generating second data 126 comprising the download request and transmitting second data 216 to core application component 204. That is, core agent component 202 can forward the download request to core application component 204. In response to receiving the download request, core application component 204 can download, via the resolver, the payload from the one or more main applications to the core application component. Specifically, core application component 204 can download the payload to a first bucket within core application component 204. Thereafter, core application component 204 can generate, via the resolver, feedback indicating status of the download operation. The feedback can be deposited by the resolver as a message in a queue within core application component 204. Based on the feedback, core agent component 202 can download the payload from the core application component 204 to the one or more agent applications.

Thus, connectivity model 110 can leverage the features of HTTP2 to enable a faster, more efficient and more secure connection between agent applications 120 and main applications 130 that can be employable to transmit requests and data between agent application 120 and main applications 130. Additional embodiments of connectivity model 110 are described in greater detail with reference to the subsequent figures.

FIG. 2 illustrates another block diagram of an example, non-limiting system 200 that can connect, via a single communication channel, one or more agent applications located within a medical device to one or more main applications external to the one or more agent applications, in accordance with one or more embodiments described herein. Repetitive description of like elements and/or processes employed in respective embodiments is omitted for sake of brevity.

Non-limiting system 200 illustrates the system of connectivity model 110. As discussed with reference to FIG. 1, connectivity model 110 can comprise core agent component 202, core application component 204, application addition component 206, and migration component 208. It should be appreciated that non-limiting systems 100 and 200 are simply intended to illustrate the interaction between agent applications 120 and main applications 130 via connectivity model 110 rather than a fixed software architecture. For example, in an embodiment, agent applications 120 and core agent component 202 can be located within a medical device, and main applications 130 and core application component 204 can be located in an external system such as a cloud environment. Similarly, in one or more embodiments, application addition component 206 can be located within the medical device, and migration component 208 can be located within the external environment.

In one or more embodiments, connectivity model 110 can employ an application protocol based on HTTP2. HTTP2 can provide the benefits of higher performance, multiplexing, server push, stream prioritization and HTTP header compression. Multiplexing can allow traffic between the client applications and the server applications to pass through a single TCP connection. TCP is a standard protocol that works with the Internet Protocol (IP). Server push is a feature in HTTP2, wherein a server (e.g., a main application) can proactively push resources onto a client (e.g., an agent application) before the client requests the resources. Stream prioritization is a feature in HTTP2 that allows clients (e.g., agent applications) to assign priority levels to different streams (e.g., requests generated by agent applications) so that high priority resources can be loaded first. HTTP header compression is a feature in HTTP2 that employs the HPACK algorithm (a header compression algorithm in HTTP2) to reduce overhead and improve performance by compressing redundant headers efficiently.

For example, in one or more embodiments, the design of the application protocol can allow multiple client applications (e.g., one or more agent applications of agent applications 120) within a medical device to communicate with their counterpart server applications (e.g., one or more main applications of main applications 130) located in an external system (e.g., a system external to the medical device) via a single communication channel (e.g., a TCP connection) and by whitelisting a single URL in the firewall at a customer's facility (e.g., a hospital, clinic or other medical facility employing the medical device). For example, core agent component 202 can wrap, via appropriate HTTP methods, requests 122 generated by one or more agent applications comprised in agent applications 120, wherein wrapping requests 122 can generate a wrapped request. Core agent component 202 can transmit the wrapped request to core application component 204 residing in an external system (e.g., a cloud computing platform such as AWS®, Microsoft Azure®, etc. or another similar external environment) via the communication channel. The wrapped request can be received by a resolver in core application component 204. The resolver can unwrap the wrapped request and accurately forward the one or more requests resulting from the unwrapping to the appropriate resource in the appropriate main application within the main applications 130. For example, based on the application ID specified in a request comprised in requests 122, the resolver can identify the main application to forward the request to, and based on the resource type specified in the request, the resolver can invoke the appropriate resource in the main application. For example, the resource type specified in a request generated by an agent application of agent application 120 can be an API, and the resolver can build a mechanism to call the API with a POST body and attest the POST body. Finally, the resolver can receive a response (e.g., feedback) back from the resource and cascade the response back to the one or more agent applications via core agent component 202.

In one or more embodiments, core agent component 202 and core application component 204 can upload/download files from one or more agent applications of agent applications 120 to/from one or more main applications of main applications 130, respectively, and the feedback associated with the respective upload and download operations can be transmitted back to the originator of the communication (in various embodiments, core agent component 202).

In one or more embodiments, the multiplexing feature of HTTP2 can provide further advantages. For example, a hospital can employ multiple medical devices (e.g., imaging devices, scanners, etc.), wherein each medical device can comprise one or more client applications that can interact with one or more server applications in an external environment via their respective connectivity model 110.

In one or more embodiments, end entities (e.g., hardware, software, machines, AI, neural networks and/or users) at hospitals, clinics or other medical facilities can observe traffic travelling from the multiple medical devices and the client applications running on those medical devices to the external environment by monitoring traffic from only one URL (associated with a core application component 204 associated with one medical device) as opposed to monitoring traffic from multiple URLs (associated with respective core application components associated with respective medical device). For example, scanner A, scanner B and scanner C can each employ a connectivity model 110 to communicate with a main applications in a cloud environment, and an end entity (e.g., hardware, software, machine, AI, neural network and/or user) can observe traffic originating from scanners A, B and C by monitoring traffic from the URL associated with core application component 204 of connectivity model 110 associated with scanner B. The multiplexing feature can also simplify the troubleshooting of connectivity issues between the client applications and the server applications. For example, an entity (e.g., hardware, software, machine, AI, neural network and/or user) can troubleshoot such connectivity issues by investigating only the connection issues related to one URL associated with a core application component 204 associated with a medical device. For example, the entity (e.g., hardware, software, machine, AI, neural network and/or user) can identify a problem originating on the URL in a firewall rule, which can simplify the investigation of connectivity issues.

In one or more embodiments, connectivity model 110 can be employed in scenarios involving high data throughput, such as those where fast response times from the cloud are desirable. In such scenarios, if the bandwidth drops during active operations, for example, due to an unstable service provider, a server application (e.g., an AI inferencing engine) in the external environment can attempt to collaborate with the corresponding client application by identifying a new pathway for communication while maintaining the quality of service. However, since the application protocol employed by connectivity model 110 can be based on HTTP2, such connectivity issues can also be mitigated because HTTP2 can ensure faster response times as compared to HTTP (or HTTP1). That is because HTTP2 can eliminate a lot of overhead from HTTP. Thus, the application protocol can also act as a mitigation point in some embodiments.

In one or more embodiments, the design of the application protocol can further allow the server applications to communicate with the designated client applications via connectivity model 110 and a server push capability of the application protocol enabled by the server push feature of HTTP2. In one or more embodiments, the design of the application protocol can further allow the client applications to prioritize streams (e.g., requests 122) transmitted to the core agent component 202 according to a priority (e.g., high, medium, low). Additionally, the design of the application protocol can allow core application component 204 to process the streams according to the assigned priority level, via the stream prioritization capability of the application protocol enabled by the stream prioritization feature of HTTP2.

In one or more embodiments, application addition component 206 can add new client applications, and the new client applications can be added without implementing changes to or updating the firewall, core agent component 202 or core application component 204. Likewise, in one or more embodiments, migration component 208 can migrate a server application from a first external environment to a second external environment (e.g., from AWS® to Microsoft Azure®), and the server application can be migrated without implementing changes to or updating the firewall, core agent component 202 or core application component 204. As a result, zero application downtime can be achieved when a new client application is added to a medical device or when a server application is migrated.

In one or more embodiments, core agent component 202 and core application component 204 can abstract the Application Layer Protocol (an Open Systems Interconnection (OSI) Layer 7 such as HTTP, File Transfer Protocol (FTP), Simple Mail Transfer Protocol (SMTP), Domain Name System (DNS), etc.) employed for communication between the agent applications 120 and main applications 130, and the communication between core agent component 202 and core application component 204 can be secured over HTTP2.

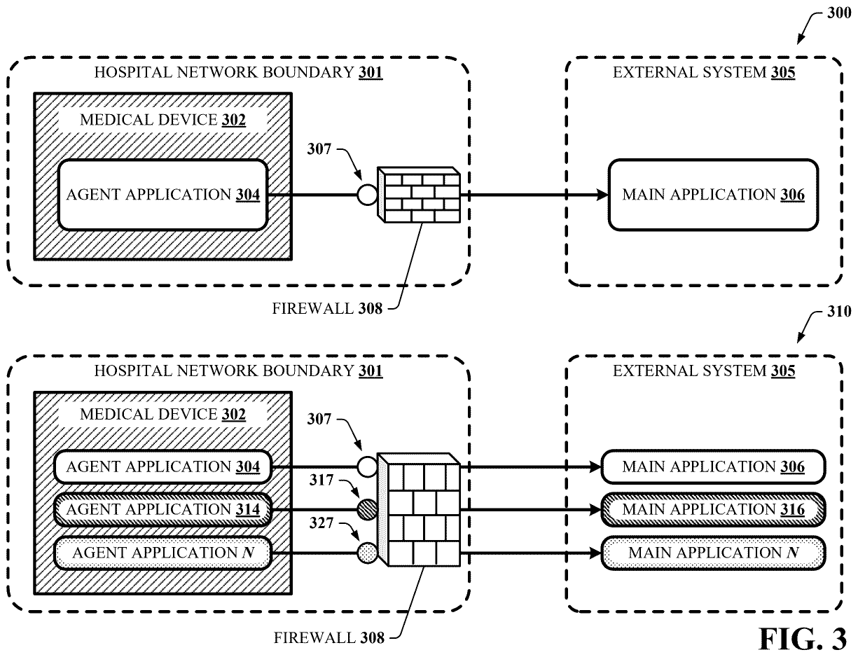

FIG. 3 illustrates block diagrams of example, non-limiting systems 300 and 310 that can connect, via multiple communication channels, one or more agent applications located within a medical device to one or more main applications external to the one or more agent applications. Repetitive description of like elements and/or processes employed in respective embodiments is omitted for sake of brevity.

FIG. 3 is intended to discuss the problems associated with onboarding software applications in a distributed architecture by employing existing systems.

Non-limiting system 300 illustrates as reference, an existing software architecture typically employed by medical facilities, wherein a portion of a software application can have a footprint on a medical device (e.g., an imaging device, a scanner, etc.) and another portion can be externally hosted on the cloud. Such software architecture is known as a distributed architecture, wherein a majority of the software applications employed by a medical device can be run on the cloud and a very small footprint of the software applications can be run on the medical device to perform simple operations such as transporting payloads from a medical device to the cloud, generating back-to-back interpretations or inferences on the medical device, etc. In non-limiting system 300, hospital network boundary 301 and external system 305 illustrate the overall blueprint of such software architecture. For example, hospital network boundary 301 can be associated with a hospital comprising medical device 302. Medical device 302 can comprise agent application 304, and external system 305 can be a cloud environment that can comprise main application 306. Agent application 304 can represent a smaller footprint of a software, and main application 306 can represent the major portion of the software dedicated to computationally heavy tasks. To execute the operations of the software, agent application 304 can communicate with main application 306 over the internet, and the communication can involve whitelisting (at 307) the URL of main application 306 on firewall 308 of the hospital's network to ensure data privacy.

Onboarding each new agent application to medical device 302 can involve whitelisting the URL of the respective main application on the firewall of the hospital's network. For example, as illustrated in non-limiting system 310, onboarding agent application 314 to medical device 302 can involve whitelisting, at 317, the URL of main application 316 on firewall 308. Similarly, onboarding agent application N to medical device 302 can involve whitelisting, at 327, the URL of main application N on firewall 308, wherein N represents a positive integer. Whitelisting a URL each time a new agent application is on boarded to a medical device can be challenging and cumbersome and present a significant bottleneck to the implementation of new software applications. This problem is exacerbated as more healthcare facilities adopt the distributed architecture illustrated in FIG. 3 for software applications in medical devices. For example, an IT administrator (admin) responsible for a software application typically has to work with hospitality staff, hospital administrators, etc. to whitelist a URL on a hospital's firewall, which can delay the onboarding of an agent application to a medical device and delay project realization, impacting the revenue streams of the software development company supplying the software application.

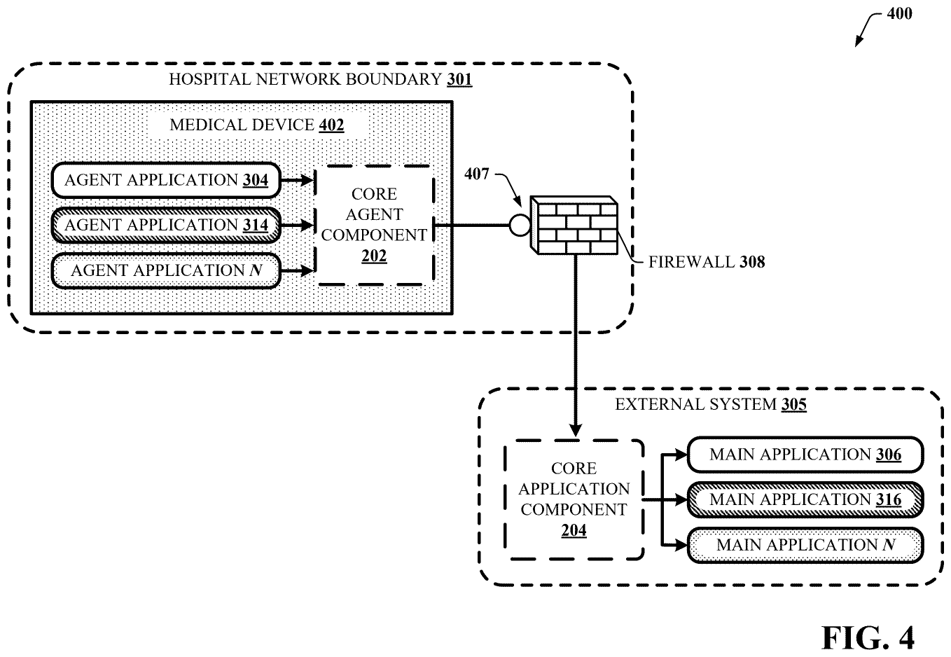

To solve the problems discussed herein in connection with distributed architectures, the inventors of the present disclosure developed the solution described by the embodiments of the present disclosure and further elaborated with reference to FIGS. 4-10.

FIG. 4 illustrates a block diagram of an example, non-limiting system 400 that can connect, via a single communication channel, one or more agent applications located within a medical device to one or more main applications external to the one or more agent applications, in accordance with one or more embodiments described herein. Repetitive description of like elements and/or processes employed in respective embodiments is omitted for sake of brevity.

With continued reference to FIG. 1-3, in one or more embodiments, core agent component 202 can be located within medical device 402 comprised in hospital network boundary 301, and core agent component 202 can interact with agent applications 304, 314, . . . , N via service discovery. Similarly, core application component 204 can be located within external system 305, and core application component 204 can interact with main applications 306, 316, . . . , N via service discovery. Recall that in software architecture, applications can interact via service discovery by dynamically locating and communicating with services in a distributed system. Agent applications 304, 314, . . . , N can be agent applications comprised in agent applications 120, and main applications 306, 316, . . . , N can be main applications comprised in main applications 130.

As described in one or more embodiments, core agent component 202 and core application component 204 can transmit requests and data between agent application 304 and main application 306, agent application 314 and main application 316, and so on, by synchronizing communication between agent applications 304, 314, . . . , N and main applications 306, 316, . . . , N, respectively, via a communication channel provided by an application protocol based on HTTP2. This can be achieved by wrapping (by core agent component 202) the requests generated by agent applications 304, 314, . . . , N into a wrapped request according to the application protocol, and transmitting (by core agent component 202) the wrapped request to core application component 204 that can then unwrap the wrapped requests by employing a resolver and route the requests comprised in the wrapped requests to the appropriate main application. For example, core application component 204 can route requests from agent application 304 to main application 306. The communication channel based on the application protocol can also allow the agent applications to interact with the respective main applications by whitelisting a single URL, for example, at 407, as opposed to whitelisting respective URLs for respective main applications. In other words, new agent applications can be installed in medical device 402 without increasing or updating the whitelisting criteria associated with firewall 308. That is, the whitelisting criteria associated with firewall 308 can remain constant.

A request generated by an agent application (e.g., agent applications 304, 314, . . . , N) can have a format wherein the agent application can specify the main Application ID, resource type, resource name, resource variables, resource method, payload data and payload data type. The main application ID can be an ID that can be well-known at the time of development or deployment of the main application. Further, the main application ID can identify the resource in the main application that an agent application intends to communicate with. The resource type can be an API, an object store (e.g., a bucket), a queue, a notification, a service name, a custom service name, a security token, user management, AI inferencing, etc. Some of the resource types can also have resource names. The resource method can specify the methods to be implemented such as, for example, GET, POST, upload, download, polling, etc. The resource methods can be optional. For example, a notification may not be associated with a resource method. Some of the resources can also optionally have resource variables. For example, when specifying GET as a resource method, the agent application can specify the device ID to get. The payload data can identify the payload that the agent application intends to transmit to its corresponding main application, and the payload data can be specified according to a payload type that can have a format such as JSON, Flat-file, XML, BLOB, JPG, PNG, etc. As described in one or more embodiments, the agent application can also assign a priority (e.g., high, medium, low) to the request by leveraging the stream prioritization feature of HTTP2. For example, agent application 304 can specify in the request that it desires to communicate with main application 306 at a high priority level.

As previously stated, in an embodiment, the resource type specified by an agent application in the request can be AI inferencing that can be accessed by an end entity (e.g., hardware, software, machine, AI, neural network and/or user) at an endpoint such as a URL or an interface. In the distributed architecture illustrated in FIG. 4 wherein agent applications 304, 314, . . . , N can be located within medical device 402 and main applications 306, 316, . . . , N can be located within external system 305, the AI inferencing can be performed in the external system 305 instead of medical device 402 because AI inferencing is resource intensive. For example, main application 306 can be an inferencing service that can perform AI processing on data received from agent application 304 and return the results of the AI processing to agent application 304. More specifically, main application 306 can be Amazon SageMaker®, a cloud-based machine learning (ML) service by AWS®. Agent application 304 can generate a request comprising AI inferencing as the resource type. For example, agent application 304 can specify the resource type as Amazon SageMaker®. Additionally, agent application 304 can specify a unique name for the Amazon SageMaker® instance and a method (if applicable) for the Amazon SageMaker® URL for the Amazon SageMaker® resource type. Agent application 304 can transmit, via connectivity model 110, the request in the JSON format to main application 306 (e.g., an AI inferencing engine) that can process the request and return, via connectivity model 110, the response to agent application 304.

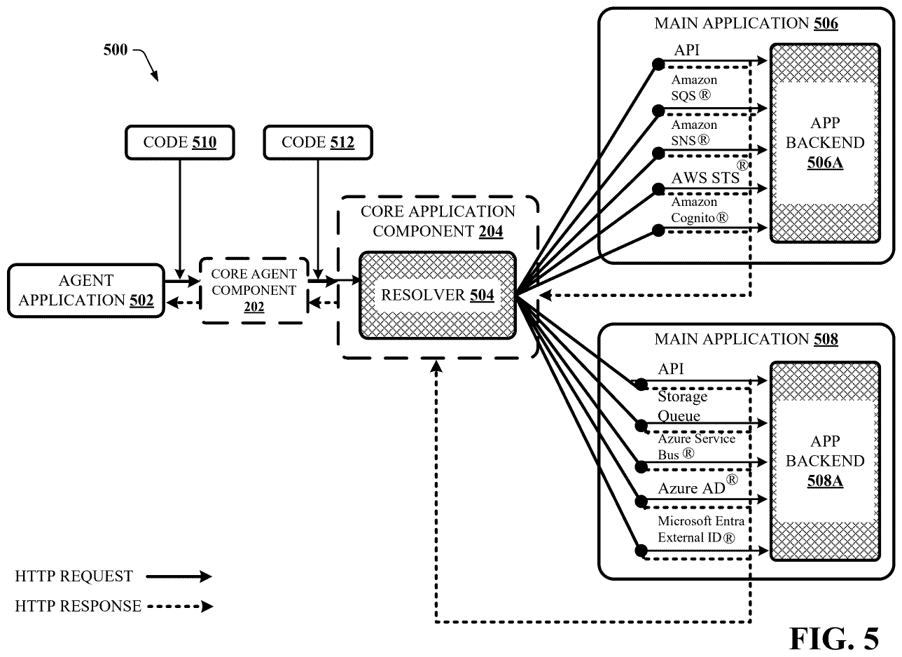

FIG. 5 illustrates a block diagram of an example, non-limiting system 500 that can transmit, via a single communication channel, a request from an agent application located within a medical device to a main application external to the agent application, in accordance with one or more embodiments described herein. Repetitive description of like elements and/or processes employed in respective embodiments is omitted for sake of brevity.

With continued reference to at least FIG. 4, agent application 502 can be an agent application comprised within agent applications 120, and main application 506 and main application 508 can be main applications comprised in main applications 130. Additionally, agent applications 120 can be located in a medical device (e.g., medical device 402), and main applications 506 and 508 can be located in a cloud environment (e.g., external system 305) external to the medical device such that agent application 502 can communicate with main application 506 or main application 508 via core agent component 202 and core application component 204. For example, main application 506 can be located in AWS® whereas main application 508 can be located in Microsoft Azure®. In an embodiment, main application 506 can be a counterpart application to agent application 502. In another embodiment, main application 508 can be a counterpart application to agent application 502. In yet another embodiment, both main applications 506 and 508 can be counterpart applications to agent application 502.

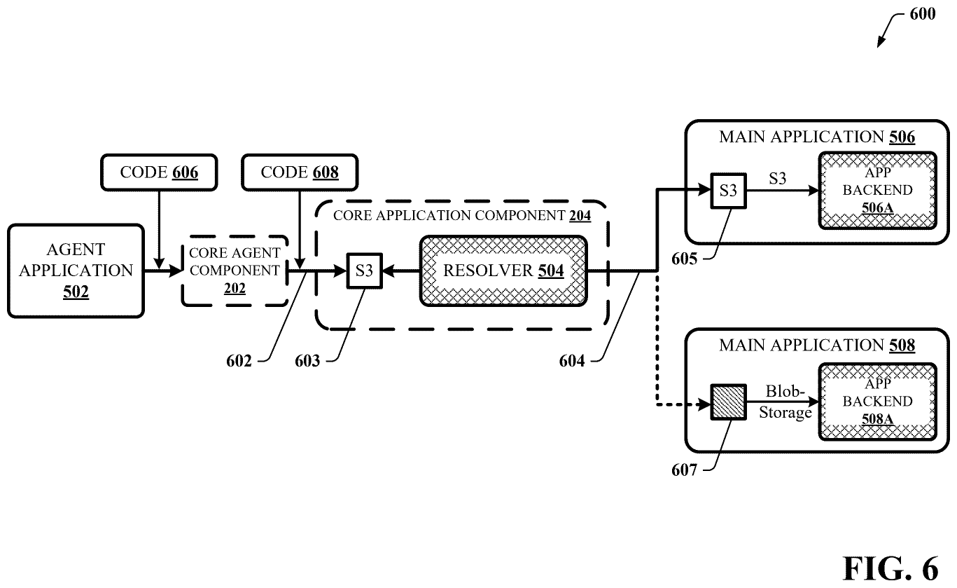

In one or more embodiments, code 510 can represent a request generated by agent application 502, as described in paragraph [00107]. Agent application 502 can transmit the request to core agent component 202. Upon receiving the request from agent application 502, core agent component 202 can first inspect the resourceType field. Unless the resourceType is specified as Amazon Simple Storage Service® (Amazon S3® or S3) by agent application 502, core agent component 202 can wrap the request from agent application 502, call the URL associated with core application component 204 and forward the request as a POST body to core application component 204. Code 512 represents the request forwarded by core agent component 202 to core application component 204. Amazon S3® is a service provided by AWS® for file uploads and downloads that allows for the storage, retrieval and management of data in the cloud. The workflows applicable to upload and download operations are described with reference to at least FIGS. 6-8.
Code 510:
{
“mainApplicationID”: “app_id_A”,
“resourceType”: “API”,
“resourceName”: “Devices”,
“resourceMethod”: “POST”,
“resourceVariables”: “”,
“payloadData”: “{“deviceName”: “ScanRoom1”, “deviceLocation”: “Bangalore”, “deviceSite”: “whitefield”, “deviceModel”: “Ascend”,
“devID”: “123”}”,
“payloadDataType”: “String”,
“priority”: “high”
}
Code 512:
URL=https://core-application
httpMethod: POST
RequestPayload:
{
“mainApplicationID”: “app_id_A”,
“resourceType”: “API”,
“resourceName”: “Devices”,
“resourceMethod”: “POST”,
“resourceVariables”: “”,
“payloadData”: “{“deviceName”: “ScanRoom1”, “deviceLocation”: “Bangalore”, “deviceSite”: “whitefield”, “deviceModel”: “Ascend”,
“devID”: “123”}”,
“payloadDataType”: “String”,
“priority”: “high”
}

As described in one or more embodiments, core application component 204 can comprise resolver 504. In an embodiment, upon receiving the request from core agent component 202, resolver 504 can inspect the POST body, extract the request originating from agent application 502, and forward the request to main application 506 and the appropriate resources of main application 506. For example, if the resourceType specified by agent application 502 is an API (resourceType=“API”), the request can be forwarded to an API. If the resourceType specified by agent application 502 is a queue (resourceType=“Queue”), the request can be forwarded to Amazon Simple Queue Service® (Amazon SQS®). If the resourceType specified by agent application 502 is a notification (resourceType=“Notification”), the request can be forwarded to Amazon Simple Notification Service® (Amazon SNS®). If the resourceType specified by agent application 502 is a security token (resourceType=“Security_Token”), the request can be forwarded to AWS Security Token Service® (AWS STS®). If the resourceType specified by agent application 502 is user management (resourceType=“User_Mgmt”), the request can be forwarded to Amazon Cognito®. The resources comprised in main application 506 can forward the request received from resolver 504 to application (app) backend 506A for further processing.

Listed below are some examples of requests with different resource types that can be generated by agent application 502 and the data associated with the requests.

If a request is for a Post API call to a main application, the agent application can transmit the following information to core agent component 202:
{
“mainApplicationID”: “app_id_A”,
“resourceType”: “API”,
“resourceName”: “Devices”,
“resourceMethod”: “POST”,
“resourceVariables”: “”,
“payloadData”: “{“deviceName”: “ScanRoom1”, “deviceLocation”: “Bangalore”, “deviceSite”: “whitefield”, “deviceModel”: “Ascend”,
“devID”: “123”}”,
“payloadDataType”: “String”,
“priority”: “high”
}

If a request is to get an API call from a main application, the agent application can transmit the following information to core agent component 202:
{
“mainApplicationID”: “app_id_A”,
“resourceType”: “API”,
“resourceName”: “Devices”,
“resourceMethod”: “GET”,
“resourceVariables”: “deviceID=123”,
“payloadData”: “”,
“payloadDataType”: “”,
“priority”: “med”
}

If a request is to upload a file to a bucket (e.g., Amazon S3®) location in a main application, the agent application can transmit the following information to core agent component 202:
{
“mainApplicationID”: “app_id_A”,
“resourceType”: “Object_Store”,
“resourceName”: “bucket_Name”,
“resourceMethod”: “upload”,
“resourceVariables”: “”,
“payloadData”: “File_Blob”,
“payloadDataType”: “Flat”,
“priority”: “low”
}

If a request is to download a file from a bucket (e.g., Amazon S3®) location in a main application, the agent application can transmit the following information to core agent component 202. The downloaded file can be sent back by core agent component 202 as a response to the agent application:
{“mainApplicationID”: “app_id_A”,
“resourceType”: “Object_Store”,
“resourceName”: “bucket_Name”,
“resourceMethod”: “download”,
“resourceVariables”: “”,
“payloadData”: “”,
“payloadDataType”: “”,
“priority”: “high”
}

If a request is to send a notification (e.g., Amazon SNS®) to a main application, the agent application can transmit the following information to core agent component 202:
{
“mainApplicationID”: “app_id_A”,
“resourceType”: “Notification”,
“resourceName”: “topicName”,
“resourceMethod”: “”,
“resourceVariables”: “”,
“payloadData”: “{“topicPayload”: “topicPayloadData”}”,
“payloadDataType”: “JSON”,
“priority”: “low”
}

If a request is to talk to any service hosted within the main application (e.g., User_mgmt(Amazon Cognito®), Security_mgmt(AWS STS®), AI_inferencing(Amazon SageMaker®), etc., the agent application can transmit the following information to core agent component 202:
{
“mainApplicationID”: “app_id_A”,
“resourceType”: “Security_token|User_Mgmt|AI_Inferencing| . . . ”,
“resourceName”: “ServiceUniqueIdentifier”,
“resourceMethod”: “MethodIfAny”,
“resourceVariables”: “VariablesIfAny”,
“payloadData”: “{“PAYLOAD_JSON”}”,
“payloadDataType”: “JSON”,
“priority”: “high”
}

If a request type is asynchronous (Async) in nature and the agent application should be notified when the response is available, the agent will embed a callback URL in the request, as follows:
{
“mainApplicationID”: “app_id_A”,
“resourceType”: “SERVICE_NAME”,
“resourceName”: “ServiceUniqueIdentifier”,
“resourceMethod”: “MethodIfAny”,
“resourceVariables”: “VariablesIfAny”,
“payloadData”: “{“PAYLOAD_JSON”}”,
“payloadDataType”: “JSON”,
“priority”: “med”,
“callBackURL”: “https://agent_url/callback_url”
}

In a different embodiment, agent application 502 can generate a request for main application 508. Accordingly, core agent component 202 can wrap the request and forward the request to core application component 204. Thereafter, resolver 504 can unwrap and forward the request to the appropriate resource in main application 508. For example, if the resourceType specified by agent application 502 is an API (resourceType=“API”), the request can be forwarded to an API. If the resourceType specified by agent application 502 is a queue (resourceType=“Queue”), the request can be forwarded to a Storage Queue. If the resourceType specified by agent application 502 is a notification (resourceType=“Notification”), the request can be forwarded to Azure Service Bus®. If the resourceType specified by agent application 502 is a security token (resourceType=“Security_Token”), the request can be forwarded to Azure Active Directory® (Azure AD). If the resourceType specified by agent application 502 is user management (resourceType=“User_Mgmt”), the request can be forwarded to Microsoft Entra External ID®. The resources comprised in main application 506 can forward the request received from resolver 504 to application (app) backend 508A for further processing.

It should be noted that the embodiments of the present disclosure were experimentally tested for proof of concept with the resourceType=“API” with successful results.

In one or more embodiments, the results and feedback generated by main application 506 or main application 508 based on the request generated by agent application 502 can be cascaded back to agent application 502 via resolver 504 and core agent component 202.

In one or more embodiments, the logic of communicating with the various resources comprised in main application 506 or main application 508 can be abstracted out from all the modules in the communication and handled only by resolver 504 (resolver module). Accordingly, resolver 504 can be designed in a scalable manner to ensure that if core application component 204 intends to interact with a new resource in the future, then the application changes can be confined to within resolver 504 without impacting the remaining modules.

FIG. 6-8 describe embodiments related to uploading and downloading files between agent application 502 and main application 506 or main application 508.

FIG. 6 illustrates a block diagram of an example, non-limiting system 600 that can transfer, via a single communication channel, data between one or more agent applications and one or more main applications external to the one or more agent applications, in accordance with one or more embodiments described herein. Repetitive description of like elements and/or processes employed in respective embodiments is omitted for sake of brevity.

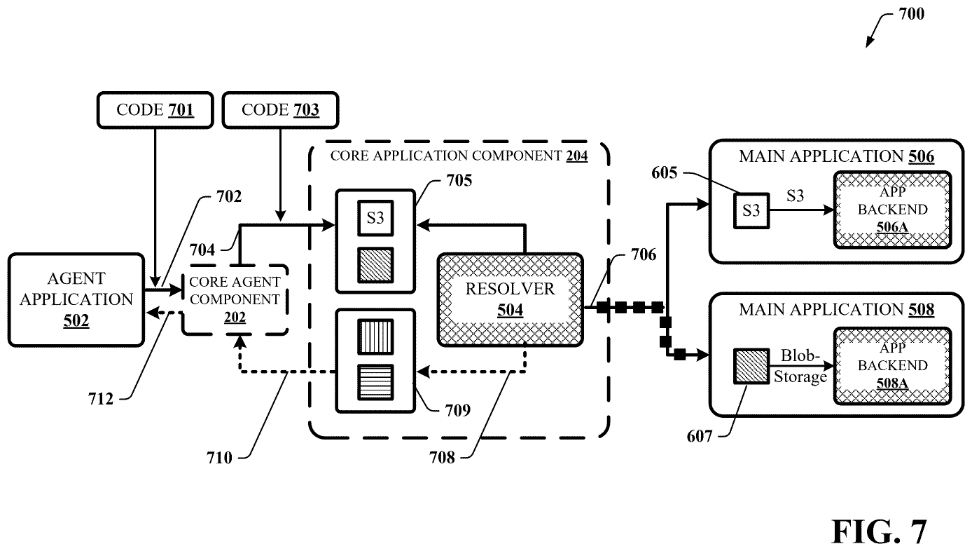

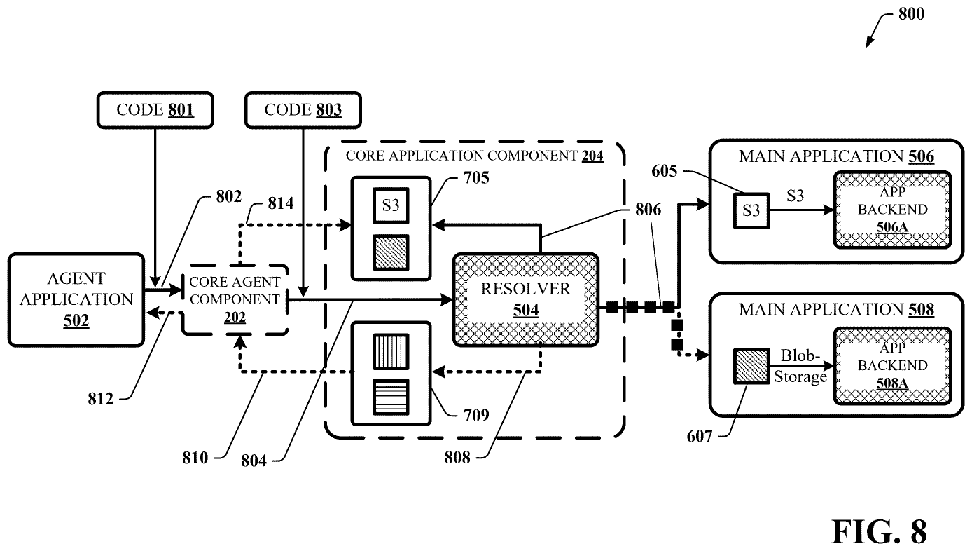

With continued reference to at least FIG. 5, non-limiting system 600 illustrates how connectivity model 110 can be employed in an upload workflow or upload operation to upload files from agent application 502 to main applications 506 and 508, or in a download workflow or download operation to download files from main applications 506 and 508 to agent applications 502. The upload and download operations are described in greater detail with reference to FIG. 7 and FIG. 8, respectively.

In one or more embodiments, the method of uploading files from agent application 502 to main application 506 or main application 508 can comprise three steps. In this regard, code 606 describes first data 124 that can be transmitted by agent application 502 to core agent component 202 to initiate the upload operation, and code 608 represents second data 126 that can be transmitted by core agent component 202 to core application component 204 to execute the upload operation. At 602, core agent component 202 can upload a file to temporary bucket 603 (e.g., an Object Store such as Amazon S3® or Blob Storage by Microsoft Azure®) within core application. At 604, resolver 504 can transfer the file from temporary bucket 603 (e.g., an Object Store such as Amazon S3®) within core application component 204 to bucket (e.g., an Object Store such as Amazon S3®) within main application 506. Alternatively, at 604, resolver 504 can transfer the file from temporary bucket 603 (e.g., an Object Store such as Blob Storage by Microsoft Azure®) within core application component 204 to bucket 607 (e.g., an Object Store such as Blob Storage by Microsoft Azure®) within main application 508. Thereafter, resolver 504 can notify core agent component 202 about the status of the upload via a message deposited in a queue within core application component 204.
Code 606 (upload operation):
{
“mainApplicationID”: “app_id_A”,
“resourceType”: “Object_Store”,
“resourceName”: “bucket_Name”,
“resourceMethod”: “upload”,
“resourceVariables”: “”,
“payloadData”: “File_Blob”,
“payloadDataType”: “Flat”
}
Code 608 (upload operation):
URL=https://core-application
httpMethod: POST
RequestPayload:
{
“mainApplicationID”: “app_id_A”,
“resourceType”: “Object_Store”,
“resourceName”: “bucket_Name”,
“resourceMethod”: “upload”,
“resourceVariables”: “”,
“payloadData”: “File_Blob”,
“payloadDataType”: “Flat”
}

In one or more embodiments, the method of downloading files from main application 506 or main application 508 to agent application 502 can comprise three steps. In this regard, code 606 describes first data 124 that can be transmitted by agent application 502 to core agent component 202 to initiate the download operation, and code 608 represents second data 126 that can be transmitted by core agent component 202 to core application component 204 to execute the download operation. At 604, resolver 504 can download the file from bucket 605 (e.g., an Object Store such as Amazon S3®) in main application 506 to temporary bucket 603 (e.g., an Object Store such as Amazon S3®). Alternatively, at 604, resolver 504 can download the file from bucket 607 (e.g., an Object Store such as Blob Storage by Microsoft Azure®) in main application 508 to temporary bucket 603 (e.g., an Object Store such as Blob Storage by Microsoft Azure®). Thereafter, resolver 504 can notify core agent component 202, via a message deposited in a queue within core application component 204, that the file is ready to be downloaded. At 602, core agent component 202 can download the file from temporary bucket 603 (e.g., an Object Store such as Amazon S3® or Blob Storage by Microsoft Azure®) and return/forward the file to agent application 502.
Code 606 (download operation):
{
“mainApplicationID”: “app_id_A”,
“resourceType”: “Object_Store”,
“resourceName”: “bucket_Name”,
“resourceMethod”: “download”,
“resourceVariables”: “”,
“payloadData”: “”,
“payloadDataType”: “”
}
Code 608 (download operation):
URL=https://core-application
httpMethod: POST

RequestPayload:
{
“mainApplicationID”: “app_id_A”,
“resourceType”: “Object_Store”,
“resourceName”: “bucket_Name”,
“resourceMethod”: “download”,
“resourceVariables”: “”,
“payloadData”: “”,
“payloadDataType”: “”
}

FIG. 7 illustrates a flow diagram of an example, non-limiting method 700 that can upload, via a single communication channel, data from one or more agent applications to one or more main applications external to the one or more agent applications, in accordance with one or more embodiments described herein. Repetitive description of like elements and/or processes employed in respective embodiments is omitted for sake of brevity.

In one or more embodiments, the upload operation to upload files from agent application 502 to main application 506 or main application 508 can comprise the following steps:

At 702, agent application 502 can initiate the upload process by communicating (e.g., via code 701) the intent to upload a file (e.g., object, payload, etc.) along with the payload (e.g., the file Blob) and a callback URL to core agent component 202, wherein the callback URL can be employed by core agent component 202 to notify/intimate agent application 502 once the upload is successful.
Code 701:
{
“mainApplicationID”: “app_id_A”,
“resourceType”: “Object_Store”,
“resourceName”: “bucket_Name”,
“resourceMethod”: “upload”,
“resourceVariables”: “”,
“payloadData”: “File_Blob”,
“payloadDataType”: “Flat”,
“priority”: “high”,
“callBackURL”: “https://agent_url/callback_url”
}

At 704, core agent component 202 can upload the file (e.g., code 703) to temporary bucket 705 (e.g., an Object Store such as Amazon S3®, Blob Storage by Microsoft Azure®, etc. depending on the choice of the cloud vendor) within core application component 204.
Code 703:
URL=https://core-application
httpMethod: POST
RequestPayload:
{
“mainApplicationID”: “app_id_A”,
“resourceType”: “Object_Store”,
“resourceName”: “bucket_Name”,
“resourceMethod”: “upload”,
“resourceVariables”: “”,
“payloadData”: “File_Blob”,
“priority”: “high”,
“payloadDataType”: “Flat”
}

At 706, resolver 504 can transfer the file from temporary bucket 705 to bucket 605 (e.g., an Object Store such as Amazon S3®). Alternatively, at 706, resolver 504 can transfer the file from temporary bucket 705 (e.g., an Object Store such as Blob Storage by Microsoft Azure®) to bucket 607 (e.g., an Object Store such as Blob Storage by Microsoft Azure®). Resolver 504 can identify the main application to transfer the file to based on the application ID specified in the file.

At 708, resolver 504 can deposit feedback in the form of a message in queue 709 for core agent component 202 to consume. The message can indicate the status of the upload operation.

At 710, core agent component 202 can read the status of the upload operation from queue 709 and proceed accordingly.

At 712, core agent component 202 can notify the status of the upload operation (e.g., “The file has been uploaded to the requested application”) to agent application 502 via the callback URL.

FIG. 8 illustrates a flow diagram of an example, non-limiting method 800 that can download, via a single communication channel, data from one or more main applications to one or more agent applications external to the one or more main applications, in accordance with one or more embodiments described herein. Repetitive description of like elements and/or processes employed in respective embodiments is omitted for sake of brevity.

In one or more embodiments, the download operation to download files from main application 506 or main application 508 to agent application 502 can comprise the following steps:

At 802, agent application 502 can communicate (e.g., via code 801) the intent to download a file (e.g., object, payload, etc.) and a callback URL to core agent component 202, wherein the callback URL can be employed by core agent component 202 to notify/intimate agent application 502 once the download is successful. Additionally, agent application 502 can identify the file to download and the bucket to download the file from.
Code 801:
“mainApplicationID”: “app_id_A”,
“resourceType”: “Object_Store”,
“resourceName”: “bucket_Name”,
“resourceMethod”: “download”,
“resourceVariables”: “”,
“payloadData”: “”,
“payloadDataType”: “”,
“priority”: “high”,
“callBackURL”: “https://agent_url/callback_url”
}

At 804, core agent component 202 can initiate the download request by communicating (e.g., via code 803) the details of the download instructions to core application component 204.
Code 803:
URL=https://core-application
httpMethod: POST
RequestPayload:
{
“mainApplicationID”: “app_id_A”,
“resourceType”: “Object_Store”,
“resourceName”: “bucket_Name”,
“resourceMethod”: “download”,
“resourceVariables”: “”,
“payloadData”: “”,
“payloadDataType”: “”,
“priority”: “high”
}

At 806, resolver 504 can transfer the file from bucket 605 (e.g., an Object Store such as Amazon S3®) to temporary bucket 705 (e.g., an Object Store such as Amazon S3®), the internal bucket of resolver 504 within core application component 204. Alternatively, at 806, resolver 504 can transfer the file from bucket 607 (e.g., an Object Store such as Blob Storage by Microsoft Azure®) to temporary bucket 705 (e.g., an Object Store such as Blob Storage by Microsoft Azure®). To download files from main application 506 or main application 508, resolver 504 can interact with each main application and their respective buckets.

At 808, resolver 504 can deposit feedback in the form of a message in queue 709 for core agent component 202 to consume. The message can indicate the status of the download operation.

At 810, core agent component 202 can read the status of the download operation from queue 709 and proceed accordingly.

At 814, core agent component 202 can download the file from temporary bucket 705.

At 812, core agent component 202 can forward the file to agent application 502 via the callback URL.

FIG. 9 illustrates a flow diagram of an example, non-limiting method 900 that can transfer, via a server push capability of an application protocol, data from one or more main applications to one or more agent applications external to the one or more main applications, in accordance with one or more embodiments described herein. Repetitive description of like elements and/or processes employed in respective embodiments is omitted for sake of brevity.

With continued reference to at least FIGS. 1 and 5, in one or more embodiments, main application 506 or main application 508 can employ the server push feature of HTTP2 via an application protocol to transmit data to agent application 902 or agent application 904 via core agent component 202 and core application component 204. For example, in certain use cases (e.g., protocol recommendation in intelligent protocolling, push protocol initiation, etc.) main application 508 can desire to send information to agent application 902 as a push notification. In one or more embodiments, employing the server push feature can comprise the following steps:

In various embodiments, to ensure data privacy, core agent component 202 can initiate a connection (e.g., open the communication channel) with core application component 204. This step (e.g., step zero) can function as a security check and an initial handshake to ensure that there is no inbound communication allowed by core agent component 202 into the medical device comprising core agent component 202.

Thereafter, at 906, main application 506 and/or main application 508 can initiate communication with core application component 204 and forward requests comprising the agent IDs of one or more agent applications that main application 506 and/or main application 508 intend to communicate with. For example, if main application 506 intends to forward JSON data to agent application 902, then main application 506 can specify the agent ID of agent application 902, the payload and the payload type in a request to core application component 204. Likewise, if main application 506 intends to forward flat data to agent application 904, then main application 506 can specify the payload data and the payload type (e.g., flat) in a request to core application component 204. Main application 506 can also include a callback URL in the request that can be employed by core application component 204 to notify main application 506 about the status of the payload (e.g., delivered, not delivered, etc.).

Request 1 describes exemplary data that can be forwarded by main application 506 and/or main application 508 to core application component 204.
Request 1:
{
“agentApplicationIDs”: [“app_id_A1”, “app_id_A2”, . . . ],
“payloadData”: “12,45,22”,
“payloadDataType”: “flat”,
“callBackURL”: “https://main_app_url/callback_url”
}

At 908, resolver 504 can forward the payload (e.g., data, package, file, object, etc.) to the appropriate core agents associated with the agent applications specified in the request and ignore the agent IDs not specified in the request. For example, agent application 902 can be located within a first medical device, and agent application 904 can be located within a second medical device that can be different from the first medical device. Additionally, both the first medical device and the second medical device can employ a connectivity model 110 to communicate with an external environment. In an embodiment, main application 506 can specify in the request to core application component 204 that it intends to communicate with agent application 902. Accordingly, resolver 504 can forward the payload to core agent component 202 associated with agent application 902. In another embodiment, main application 506 can specify in the request to core application component 204 that it intends to communicate with agent application 904. Accordingly, resolver 504 can forward the payload to core agent component 202 associated with agent application 904.

At 910, core agent component 202 can deliver the payload to the intended agent application. For example, core agent component 202 associated with agent application 902 can deliver the payload to agent application 902. Likewise, core agent component 202 associated with agent application 904 can deliver the payload to agent application 904.

At 912, the respective core agent components can acknowledge the delivery of the payload via a message to core application component 204.

At 914, resolver 504 can employ the callback URL to notify main application 506 and/or main application 508 that the payload has been delivered to the intended agent applications.

FIG. 10 illustrates a block diagram of an example, non-limiting system 1000 that can transmit, via a stream prioritization capability of an application protocol, one or more requests from one or more agent applications located within a medical device to one or more main applications external to the one or more agent applications, in accordance with one or more embodiments described herein. Repetitive description of like elements and/or processes employed in respective embodiments is omitted for sake of brevity.

With continued reference to at least FIGS. 1 and 5, in one or more embodiments, agent application 1002, agent application 1004 and agent application 1006 can employ the stream prioritization feature of HTTP2 via an application protocol to assign priorities to one or more requests.

For example, at 1007, agent application 1002, agent application 1004 and agent application 1006 can inform core agent component 202 about their respective priorities. For example, agent application 1002 can assign a high priority to a request transmitted by agent application 1002 to core agent component 202, agent application 1004 can assign a medium priority to a request transmitted by agent application 1004 to core agent component 202, and agent application 1006 can assign a low priority to a request transmitted by agent application 1006 to core agent component 202.

At 1008, core agent component 202 can leverage the stream priority feature of HTTP2 to transmit the requests from agent application 1002, 1004 and 1006 to core application component 204 according to the respective priorities (e.g., high, medium, low) assigned to the respective requests.

Thereafter, core application component 204 can leverage the stream prioritization feature of HTTP2 to read and process the request streams according to their priorities by employing resolver 504. For example, core application component 204 can comprise a high priority queue for high priority requests, a medium priority queue for medium priority requests and a low priority queue for low priority requests. In non-limiting system 1000, the priority levels are described by the legend inside the box of resolver 504.

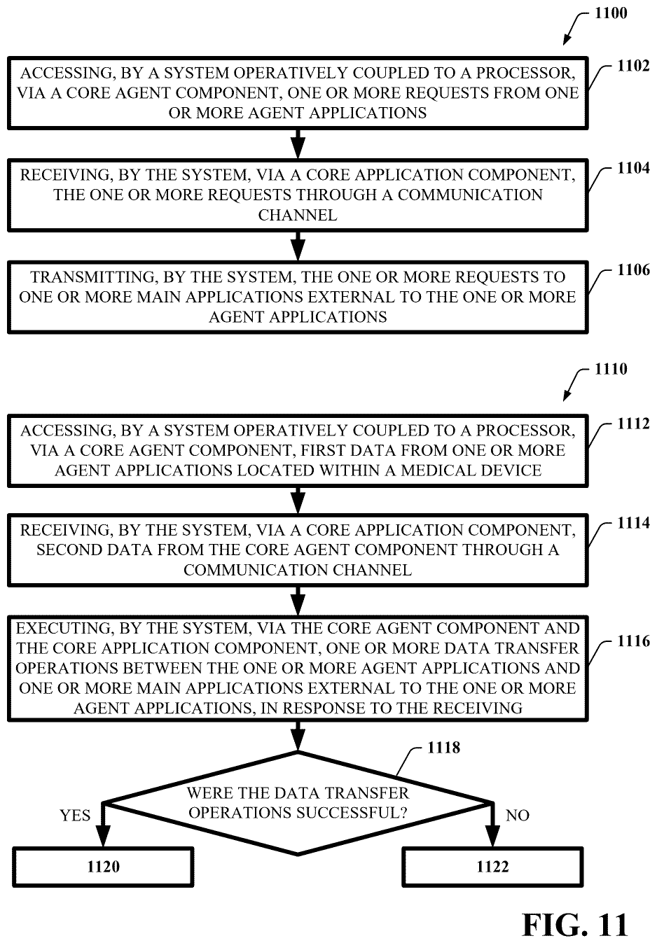

FIG. 11 illustrates flow diagrams of example, non-limiting methods 1100 and 1110 that can connect agent applications located within a medical device to their corresponding main applications via a common communication channel, in accordance with one or more embodiments described herein. Repetitive description of like elements and/or processes employed in respective embodiments is omitted for sake of brevity.

Non-limiting method 1100 can be employed to transmit requests and data between the agent applications and the main applications.

At 1102, non-limiting method 1100 can comprise accessing (e.g., by core agent component 202), by a system operatively coupled to a processor, via a core agent component, and one or more requests from one or more agent applications.

At 1104, non-limiting method 1100 can comprise accessing (e.g., by core application component 204), by the system, via a core agent component, one or more requests from one or more agent applications.

At 1106, non-limiting method 1100 can comprise transmitting (e.g., by core application component 204), by the system, the one or more requests to one or more main applications external to the one or more agent applications.

Non-limiting method 1110 can be employed to upload/download files from the agent applications to/from the main applications, respectively.

At 1112, non-limiting method 1110 can comprise accessing (e.g., by core agent component 202), by a system operatively coupled to a processor, via a core agent component, first data from one or more agent applications located within a medical device.

At 1114, non-limiting method 1110 can comprise receiving (e.g., by core application component 204), by the system, via a core application component, and second data from the core agent component through a communication channel.

At 1116, non-limiting method 1110 can comprise executing (e.g., by core application component 204), by the system, via the core agent component and the core application component, one or more data transfer operations between the one or more agent applications and one or more main applications external to the one or more agent applications, in response to the receiving.

At 1118, non-limiting method 1110 can comprise determining (e.g., by core application component 204), by the system, whether the data transfer operations are successful.

If yes, then at 1120, non-limiting method 1110 can comprise generating (e.g., by core application component 204), by the system, feedback indicative of the successful data transfer operations and transmitting the feedback to the appropriate agent applications or main applications.

If not, then at 1122, non-limiting method 1110 can comprise generating (e.g., by core application component 204), by the system, feedback indicative of the unsuccessful data transfer operations and transmitting the feedback to the appropriate agent applications or main applications.

In various instances, machine learning algorithms or models can be implemented in any suitable way to facilitate any suitable aspects described herein. To facilitate some of the above-described machine learning aspects of various embodiments, consider the following discussion of artificial intelligence (AI). Various embodiments described herein can employ AI to facilitate automating one or more features or functionalities. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute) described herein, components described herein can examine the entirety or a subset of the data to which it is granted access and can provide for reasoning about or determine states of the system or environment from a set of observations as captured via events or data. Determination can be employed to identify a specific context or action, or can generate a probability distribution over states, for example. The determinations can be probabilistic; that is, the computation of a probability distribution over states of interest based on a consideration of data and events. Determination can also refer to techniques employed for composing higher-level events from a set of events or data.

Such determinations can result in the construction of new events or actions from a set of observed events or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes or systems (e.g., support vector machines, neural networks, expert systems, Bayesian belief networks, fuzzy logic, data fusion engines, and so on) in connection with performing automatic or determined action in connection with the claimed subject matter. Thus, classification schemes or systems can be used to automatically learn and perform a number of functions, actions, or determinations.

A classifier can map an input attribute vector, z=(z₁, z₂, z₃, z₄, zₙ), to a confidence that the input belongs to a class, as by f(z)=confidence(class). Such classification can employ a probabilistic or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, or probabilistic classification models providing different patterns of independence, any of which can be employed. Classification as used herein also is inclusive of statistical regression that is utilized to develop models of priority.

In order to provide additional context for various embodiments described herein, FIG. 12 and the following discussion are intended to provide a brief, general description of a suitable computing environment 1200 in which the various embodiments described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and include any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 12, the example environment 1200 for implementing various embodiments of the aspects described herein includes a computer 1202, the computer 1202 including a processing unit 1204, a system memory 1206 and a system bus 1208. The system bus 1208 couples system components including, but not limited to, the system memory 1206 to the processing unit 1204. The processing unit 1204 can be any of various commercially available processors. Dual microprocessors and other multi-processor architectures can also be employed as the processing unit 1204.

The system bus 1208 can be any of several types of bus structure that can further interconnect to a memory bus (with or without a memory controller), a peripheral bus, and a local bus using any of a variety of commercially available bus architectures. The system memory 1206 includes ROM 1210 and RAM 1212. A basic input/output system (BIOS) can be stored in a non-volatile memory such as ROM, erasable programmable read only memory (EPROM), EEPROM, which BIOS contains the basic routines that help to transfer information between elements within the computer 1202, such as during startup. The RAM 1212 can also include a high-speed RAM such as static RAM for caching data.

The computer 1202 further includes an internal hard disk drive (HDD) 1214 (e.g., EIDE, SATA), one or more external storage devices 1216 (e.g., a magnetic floppy disk drive (FDD) 1216, a memory stick or flash drive reader, a memory card reader, etc.) and a drive 1220, e.g., such as a solid state drive, an optical disk drive, which can read or write from a disk 1222, such as a CD-ROM disc, a DVD, a BD, etc. Alternatively, where a solid-state drive is involved, disk 1222 would not be included, unless separate. While the internal HDD 1214 is illustrated as located within the computer 1202, the internal HDD 1214 can also be configured for external use in a suitable chassis (not shown). Additionally, while not shown in environment 1200, a solid-state drive (SSD) could be used in addition to, or in place of, an HDD 1214. The HDD 1214, external storage device(s) 1216 and drive 1220 can be connected to the system bus 1208 by an HDD interface 1224, an external storage interface 1226 and a drive interface 1228, respectively. The interface 1224 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1202, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1212, including an operating system 1230, one or more application programs 1232, other program modules 1234 and program data 1236. All or portions of the operating system, applications, modules, or data can also be cached in the RAM 1212. The systems and methods described herein can be implemented utilizing various commercially available operating systems or combinations of operating systems.

Computer 1202 can optionally comprise emulation technologies. For example, a hypervisor (not shown) or other intermediary can emulate a hardware environment for operating system 1230, and the emulated hardware can optionally be different from the hardware illustrated in FIG. 12. In such an embodiment, operating system 1230 can comprise one virtual machine (VM) of multiple VMs hosted at computer 1202. Furthermore, operating system 1230 can provide runtime environments, such as the Java runtime environment or the .NET framework, for applications 1232. Runtime environments are consistent execution environments that allow applications 1232 to run on any operating system that includes the runtime environment. Similarly, operating system 1230 can support containers, and applications 1232 can be in the form of containers, which are lightweight, standalone, executable packages of software that include, e.g., code, runtime, system tools, system libraries and settings for an application.

Further, computer 1202 can be enabled with a security module, such as a trusted processing module (TPM). For instance, with a TPM, boot components hash next in time boot components and wait for a match of results to secured values, before loading a next boot component. This process can take place at any layer in the code execution stack of computer 1202, e.g., applied at the application execution level or at the OS kernel level, thereby enabling security at any level of code execution.

A user can enter commands and information into the computer 1202 through one or more wired/wireless input devices, e.g., a keyboard 1238, a touch screen 1240, and a pointing device, such as a mouse 1242. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1204 through an input device interface 1244 that can be coupled to the system bus 1208, but can be connected by other interfaces, such as a parallel port, an IEEE 1394 serial port, a game port, a USB port, an IR interface, a BLUETOOTH® interface, etc.

A monitor 1246 or other type of display device can be also connected to the system bus 1208 via an interface, such as a video adapter 1248. In addition to the monitor 1246, a computer typically includes other peripheral output devices (not shown), such as speakers, printers, etc.

The computer 1202 can operate in a networked environment using logical connections via wired or wireless communications to one or more remote computers, such as a remote computer(s) 1250. The remote computer(s) 1250 can be a workstation, a server computer, a router, a personal computer, portable computer, microprocessor-based entertainment appliance, a peer device or other common network node, and typically includes many or all of the elements described relative to the computer 1202, although, for purposes of brevity, only a memory/storage device 1252 is illustrated. The logical connections depicted include wired/wireless connectivity to a local area network (LAN) 1254 or larger networks, e.g., a wide area network (WAN) 1256. Such LAN and WAN networking environments are commonplace in offices and companies, and facilitate enterprise-wide computer networks, such as intranets, all of which can connect to a global communications network, e.g., the Internet.

When used in a LAN networking environment, the computer 1202 can be connected to the local network 1254 through a wired or wireless communication network interface or adapter 1258. The adapter 1258 can facilitate wired or wireless communication to the LAN 1254, which can also include a wireless access point (AP) disposed thereon for communicating with the adapter 1258 in a wireless mode.

When used in a WAN networking environment, the computer 1202 can include a modem 1260 or can be connected to a communications server on the WAN 1256 via other means for establishing communications over the WAN 1256, such as by way of the Internet. The modem 1260, which can be internal or external and a wired or wireless device, can be connected to the system bus 1208 via the input device interface 1244. In a networked environment, program modules depicted relative to the computer 1202 or portions thereof can be stored in the remote memory/storage device 1252. It will be appreciated that the network connections shown are examples and other means of establishing a communications link between the computers can be used.

When used in either a LAN or WAN networking environment, the computer 1202 can access cloud storage systems or other network-based storage systems in addition to, or in place of, external storage devices 1216 as described above, such as, but not limited to a network virtual machine providing one or more aspects of storage or processing of information. Generally, a connection between the computer 1202 and a cloud storage system can be established over a LAN 1254 or WAN 1256 e.g., by the adapter 1258 or modem 1260, respectively. Upon connecting the computer 1202 to an associated cloud storage system, the external storage interface 1226 can, with the aid of the adapter 1258 or modem 1260, manage storage provided by the cloud storage system as it would other types of external storage. For instance, the external storage interface 1226 can be configured to provide access to cloud storage sources as if those sources were physically connected to the computer 1202.

The computer 1202 can be operable to communicate with any wireless devices or entities operatively disposed of in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

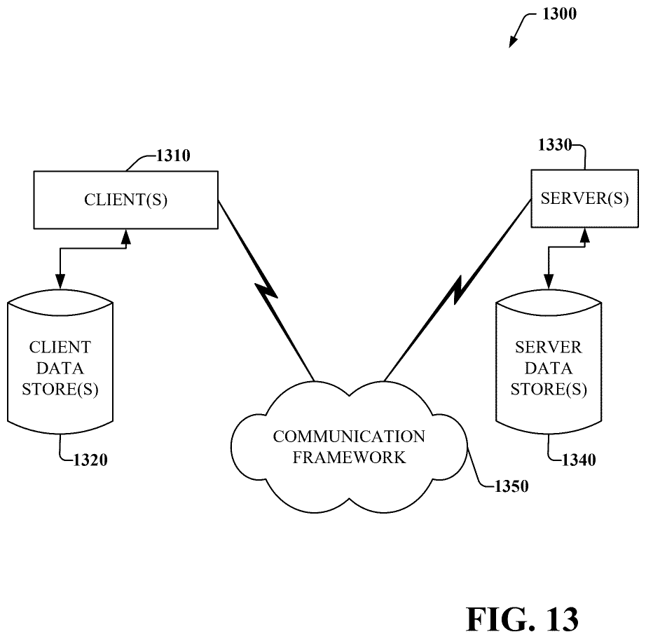

FIG. 13 is a schematic block diagram of a sample computing environment 1300 with which the disclosed subject matter can interact. The sample computing environment 1300 includes one or more client(s) 1310. The client(s) 1310 can be hardware or software (e.g., threads, processes, computing devices). The sample computing environment 1300 also includes one or more server(s) 1330. The server(s) 1330 can also be hardware or software (e.g., threads, processes, computing devices). The servers 1330 can house threads to perform transformations by employing one or more embodiments as described herein, for example. One possible communication between a client 1310 and a server 1330 can be in the form of a data packet adapted to be transmitted between two or more computer processes. The sample computing environment 1300 includes a communication framework 1350 that can be employed to facilitate communications between the client(s) 1310 and the server(s) 1330. The client(s) 1310 are operably connected to one or more client data store(s) 1320 that can be employed to store information local to the client(s) 1310. Similarly, the server(s) 1330 are operably connected to one or more server data store(s) 1340 that can be employed to store information local to the servers 1330.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can be executed from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architecture such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random-access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration but are not intended to be exhaustive or limited to the embodiments disclosed. Many modifications and variations will be apparent without departing from the scope and spirit of the embodiments described. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer-executable components; and
a processor that executes the computer-executable components stored in the memory, wherein the computer-executable components comprise:
a core agent component that accesses one or more requests from one or more agent applications; and
a core application component that:
receives, via a communication channel, the one or more requests from the core agent component; and
transmits the one or more requests to one or more main applications external to the one or more agent applications, wherein the communication channel is based on an application protocol that allows multiple requests to be transmitted from the one or more agent applications to the one or more main applications, via the core agent component and the core application component, by whitelisting only one Uniform Resource Locator (URL) on a firewall associated with the core application component.

2. The system of claim 1, wherein the one or more agent applications are located within a medical imaging device, and wherein the one or more main applications are located in a first external system.

3. The system of claim 2, wherein the core application component transmits data from the one or more main applications to the one or more agent applications, via the core agent component, by employing a server push capability of the application protocol.

4. The system of claim 2, wherein the core agent component prioritizes transmission of respective requests comprised in the one or more requests by employing a stream prioritization capability of the application protocol.

5. The system of claim 1, wherein transmitting the one or more requests to the one or more main applications comprises:
wrapping, by the core agent component, the one or more requests, wherein the wrapping generates a wrapped request; and
forwarding, by the core agent component, the wrapped request to the core application component.

6. The system of claim 5, wherein the transmitting the one or more requests to the one or more main applications further comprises:
accessing, by the core application component, the wrapped request;
unwrapping, by the core application component, the wrapped request; and
forward, by the core application component, the wrapped request to the one or more main applications.

7. The system of claim 6, wherein the transmitting the one or more requests to the one or more main applications further comprises:
accessing, by the core application component, feedback associated with the routing from the one or more main applications; and
transmitting, by the core application component, the feedback to the one or more agent applications via the core agent component.

8. The system of claim 2, wherein the computer-executable components further comprise:
an application addition component that adds at least one new agent application to the medical imaging device, wherein adding the at least one new agent application to the medical imaging device does not involve updating the firewall and application downtime.

9. The system of claim 2, wherein the computer-executable components further comprise:
a migration component that migrates at least one main application of the one or more main applications from the first external system to a second external system, wherein migrating the at least one main application to the second external system does not involve updating the firewall and application downtime.

10. The system of claim 8, wherein the URL is employable by an end entity to monitor traffic from the medical imaging device and one or more additional medical imaging devices by monitoring only the traffic associated with the URL.

11. The system of claim 1, wherein transmitting the one or more requests over the communication channel increases speed of transmission and simplifies troubleshooting of connectivity issues.

12. A computer-implemented method, comprising:
accessing, by a system operatively coupled to a processor, via a core agent component, first data from one or more agent applications located within a medical device;
receiving, by the system, via a core application component, second data from the core agent component through a communication channel; and
executing, by the system, via the core agent component and the core application component, one or more data transfer operations between the one or more agent applications and one or more main applications external to the one or more agent applications, in response to the receiving.

13. The computer-implemented method of claim 12, wherein the first data comprises an intent of the one or more agent applications to upload a payload to the one or more main applications, the payload and a callback URL, wherein the second data comprises the payload, and wherein the one or more data transfer operations comprise an upload operation to transfer the payload from the one or more agent applications to the one or more main applications.

14. The computer-implemented method of claim 13, wherein uploading the payload to the one or more main applications comprises:
uploading, by the system, via the core agent component, the payload to the core application component;
uploading, by the system, via the core application component, the payload from the core application component to the one or more main applications, in response to the core application component receiving the payload;
generating, by the system, via the core application component, feedback indicating status of the upload operation; and
transmitting, by the system, via the core agent component, the feedback from the core application component to the one or more agent applications by employing the callback URL.

15. The computer-implemented method of claim 12, wherein the first data comprises an intent of the one or more agent applications to download a payload from the one or more main applications and a callback URL, wherein the second data comprises a download request, and wherein the one or more data transfer operations comprise a download operation to transfer the payload from the one or more main applications to the one or more agent applications.

16. The computer-implemented method of claim 15, wherein downloading the payload to the one or more agent applications comprises:
forwarding, by the system, via the core agent component, the download request to the core application component;
downloading, by the system, via the core application component, the payload from the one or more main applications to the core application component, in response to the core application component receiving the download request;
generating, by the system, via the core application component, feedback indicating status of the download operation; and
downloading, by the system, via the core agent component, the payload from the core application component to the one or more agent applications, based on the feedback.

17. The computer-implemented method of claim 12, wherein the communication channel is based on an application protocol that allows data to be transferred between the one or more agent applications and the one or more main applications, via the core agent component and the core application component, by whitelisting a URL on a firewall associated with the core application component.

18. The computer-implemented method of claim 17, wherein transmitting the data over the communication channel increases a speed of transmission and simplifies troubleshooting of connectivity issues.

19. A computer program product comprising a non-transitory computer readable memory having program instructions embodied therewith, the program instructions executable by a processor to cause the processor to:
access, via a core agent component, one or more requests from one or more agent applications;
receive, via a core application component, the one or more requests from the core agent component through a communication channel; and
transmit, via the core application component, the one or more requests to one or more main applications external to the one or more agent applications.

20. The computer program product of claim 19, wherein the program instructions are further executable by the processor to cause the processor to:
wrap, via the core agent component, the one or more requests, wherein wrapping the one or more requests generates a wrapped request;
forward, via the core agent component, the wrapped request to a resolver;
access, via the core application component, the wrapped request;
unwrap, via the core application component, the wrapped request; and
forward, via the core application component, the wrapped request to the one or more main applications.
